(19) United States
(12) Patent Application Publication
(10) Pub. No.: US 2020/0246604 A1
(43) Pub. Date: Aug. 6, 2020

(54) FLUID DRAINAGE OR DELIVERY DEVICE FOR TREATMENT SITE

(71) Applicant: AROA BIOSURGERY LIMITED, Auckland (NZ)

(72) Inventors: Dorrin ASEFI, Auckland (NZ); Samuel CUTAJAR, Auckland (NZ); Alister Todd JOWSEY, Auckland (NZ); Isaac Tristram Tane MASON, Auckland (NZ); Russell Leith SPEIDEN, Auckland (NZ); Elliot Graham THOMPSON-BEAN, Auckland (NZ); William Andrew WALBRAN, Auckland (NZ); Brian Roderick WARD, Waiau Pa (NZ)

(21) Appl. No.: 16/753,725

(22) PCT Filed: Oct. 3, 2018
(86) PCT No.: PCT/NZ2018/050134

§ 371 Date: Apr. 3, 2020

Related U.S. Application Data
(60) Provisional application No. 62/679,207, filed on Jun. 1, 2018.
(60) Provisional application No. 62/568,914, filed on Oct. 6, 2017.

(51) Int. Cl. A61M 39/02; A61M 1/00; A61M 3/02; A61L 29/00; A61L 29/14; A61L 29/16

(52) U.S. Cl. CPC A61M 39/0247; A61M 1/008; A61M 3/0279; A61L 29/005; A61L 29/148; A61M 2039/082; A61M 2039/0261; A61M 2039/0264; A61M 2039/0276; A61M 2039/0282; A61L 29/16

(57) ABSTRACT

A bioresorbable device (2901) for implantation at a treatment site in the body of a patient, for draining fluid from the treatment site or delivering fluid to the treatment site. The device has a bioresorbable resilient truss (2915, 2916) for holding two tissue surfaces spaced apart, thereby defining a channel into which fluid from the treatment site can drain or from which fluid can be delivered to the treatment site, and a port in fluid communication with the one or more channels. The port is connectable to a source of negative pressure or positive pressure.

### TECHNICAL FIELD

The invention relates to a device for implanting at a treatment site for the drainage of fluid from the site or for the delivery of fluid to the site. In particular, the device is bioresorbable. The invention also relates to a system comprising the device and a means for applying negative or positive pressure to aid in reducing dead space and improve drainage of fluid from a treatment site or delivery of fluid to a treatment site. The invention further relates to a method of draining fluid from a treatment site or delivering fluid to a treatment site using the device of the invention, and to a method of manufacturing said device.

### BACKGROUND OF THE INVENTION

The drainage of fluid and the reduction of dead space from surgical or traumatic wounds is often a critical factor in the timely and effective recovery of a patient. Currently, there is no good solution for eliminating dead space at the time of surgery. Suturing provides linear closure rather than offering closure across the entire separated tissue plane. Surgical drains are only partially effective in removing fluid and do not deal with the primary issue of closing dead space immediately following surgery. Tissue adhesives have not proven to be reliably effective, and manually suturing across a total area only provides limited amount of localized closure.

Seroma or hematoma formation post-surgery or trauma can hinder recovery. Seromas and hematomas are pockets of serous fluid or blood that accumulate at wound sites. In the absence of adequate drainage, poor healing, infection or dehiscence may lead to a requirement for additional surgery and longer hospital stays. Seromas and hematomas are common after reconstructive plastic surgery procedures, trauma, mastectomy, tumour excision, caesarean, hernia repair and open surgical procedures involving a lot of tissue elevation and separation.

While reducing dead space and providing drainage of fluid from a wound site is highly desirable in many instances, it is useful in other circumstances to be able to deliver fluid directly to a wound site to aid in the wound healing process. For example, instilling antimicrobial solutions locally into infected tissue is useful for managing infections. Similarly, instillation of local anaesthetics can aid pain management.

Numerous devices are available which can be implanted at the site of treatment to enable drainage of fluid. These range from simple silicon tubes comprising drainage holes through to manifolds of structures of various shapes made from decellularised tissue. For example, U.S. Pat. No. 7,699,831 describes a wound drainage assembly having a housing configured for placement in an interior wound site. A foam sponge is located in the housing for absorbing fluid from the wound site. Tubing is coupled to the housing and is connected to a source of negative pressure outside the body. The negative pressure causes the fluid to flow from the foam sponge to an external collection site.

Some drainage devices must be removed from the body after the wound site has been drained for a period of time. Removal of such devices can cause discomfort or pain for the patient or require an undesirable further surgical procedure, while the need to remove the device limits the ability to position the device to provide effective treatment across an area. However, other drainage devices are constructed of a material capable of being absorbed by the body.

US 2015/0320911 describes tissue-based implantable drainage manifolds. The manifolds may comprise decellularised tissue formed into sheets, tubes or columns. Negative pressure may be applied to assist drainage from the wound site into the manifold and to the exterior of the body via tubing. The tissue-based manifolds do not need to be removed following completion of the drainage procedure. The manifold structures also provide a scaffold for the migration and proliferation of cells from surrounding native tissue.

However, while a number of existing drainage structures are bioresorbable their construction typically involves materials which are completely synthetic and are constructed using manufacturing techniques such as injection moulding or extrusion which create continuous tubes or structures comprising thick wall sections or structures with a high amount of synthetic mass.

The implantation of synthetic materials can contribute to elevated levels of inflammation that typically manifest within the body following implantation, most particular in sensitive and vascular areas such as the pelvic floor or abdominal wall. Many bioresorbable materials also degrade and resorb through a process of bulk hydrolysis where the polymer chains of the synthetic material absorb water to break down the chemical structure to the various monomers which release harmful acids that can trigger elevated inflammation and a foreign body response such as seen with synthetic meshes commonly used in hernia abdominal wall repair and pelvic organ prolapse repair.

It is therefore an object of the invention to provide a fluid drainage or delivery device that addresses one or more of the abovementioned shortcomings, and/or at least to provide a useful alternative to existing devices.

In this specification where reference has been made to patent specifications, other external documents, or other sources of information, this is generally to provide a context for discussing features of the invention. Unless specifically stated otherwise, reference to such external documents or sources of information is not to be construed as an admission that such documents or such sources of information, in any jurisdiction, are prior art or form part of the common general knowledge in the art.

### SUMMARY OF INVENTION

In a first aspect, the present invention provides a bioresorbable device for implantation at a treatment site in the body of a patient for draining fluid from the treatment site or delivering fluid to the treatment site. The device comprises a bioresorbable resilient truss for holding two tissue surfaces spaced apart, thereby defining a channel into which fluid from the treatment site can drain or from which fluid can be delivered to the treatment site, and a port in fluid communication with the one or more channels and being connectable to a source of negative pressure or positive pressure.

In an embodiment, the truss comprises a flexible elongate truss member. The truss may be curved and located along a wall of the channel.

In an embodiment, the truss member is substantially helical.

The truss may define the channel. For example, the outer diameter of a substantially cylindrical helical truss may correspond to the diameter of the channel. Or the width of a truss may correspond to the width of the channel. In an embodiment, the truss forms a flexible tube defining the channel. The tube may be substantially cylindrical or oval or elliptical or otherwise shaped. In an embodiment, the truss has a substantially circular cross-section in a resting, non-implanted state, and takes on an oval or elliptical cross section upon implantation in response to compressive forces acting on the truss, to define a channel with a correspondingly oval or elliptical cross-section.

The device may comprise a plurality of flexible elongate truss members. In one embodiment, a first one of the truss members is substantially helical with a first pitch length, and a second one of the truss members is substantially helical with a second pitch length. In an embodiment, the second pitch length is different to the first pitch length. For example, the first pitch length may be between about three to about five times the second pitch length, preferably about 4.5 times the second pitch length. Alternatively the first pitch length and the second pitch length may be the same, and the two respective truss members wound in opposite directions. The first and second truss members may be joined together and/or to bracing members at discrete points.

The channel may be circular in cross section, or non-circular, for example oval or elliptical.

The device may comprise two flexible elongate side truss members, each extending longitudinally along a side of the channel and joined at discrete points to the first and/or second truss members. The truss members may be joined by heat welding, stitching, or by adhesive, as examples. In an embodiment having an oval or elliptical cross sectional profile, the flexible elongate side truss members may be provided at on the minor axis of the cross section. In one embodiment, the device comprises two pairs of elongate side truss members, running along opposite sides of the truss.

The device may further comprise a flexible bioresorbable sheet, the sheet forming at least a portion of a wall of the channel. In an embodiment, the channel is formed between a surface of the flexible sheet and the surface of tissue or bone of the treatment site. For example, the sheet may be laid over an arch-type truss member. Alternatively, the flexible sheet may be wrapped around the truss, for example to enclose the truss. A plurality of apertures may be provided in the flexible sheet along a wall of the channel to permit fluid flow into the channel. The apertures may be provided as one or more rows of regularly spaced apertures, or irregularly arranged. The apertures may only be provided in selected portions of the device to selectively drain fluid from or deliver fluid to target areas of the treatment site.

In an embodiment, the device comprises two flexible bioresorbable sheets, wherein the channel is formed between facing surfaces of the two flexible sheets. The sheets may be stitched or adhered together along side seams. A plurality of apertures may be provided in one or both flexible sheets along a wall of the channel to permit fluid flow into the channel. The apertures may be provided as one or more rows of regularly spaced apertures, or irregularly arranged. The apertures may only be provided in selected portions of the device to selectively drain fluid from or deliver fluid to target areas of the treatment site.

In an embodiment, at least one truss member may comprise a length of thread or tape woven or sewn into or through at least one flexible sheet. For example, filament/thread sewn using a zig-zag stitch through one or more layers of flexible sheet. In an embodiment, the truss member(s) comprise suture.

In an embodiment, the or each flexible sheet comprises one or more layers of extracellular matrix (ECM) or polymeric material. The ECM may be formed from decellularised propria-submucosa of a ruminant forestomach. The ECM may contain a bioactive agent selected from the group comprising doxycycline, tetracyclines, silver, FGF-2, TGF-B, TGF-B2, BMR7, BMP-12, PDGF, IGF, collagen, elastin, fibronectin, and hyaluronan.

In an embodiment, the truss forms an elongate flexible tube defining the channel, and the device comprises one or more joiners holding at least a length of the flexible tube in a sinuous shape. Alternatively or additionally, the truss may define a plurality of channels into which fluid from the treatment site can drain or from which fluid can be delivered to the treatment site. For example the truss may define a primary channel and a plurality of secondary channels branching off the primary channel.

In an embodiment, the treatment site is a space between surfaces of muscle tissue, connective tissue or skin tissue that have been separated during surgery or as a result of trauma.

In an embodiment the treatment site is an exposed area of tissue, such as muscle or subcutaneous tissue, in an open surgical or tunnelled wound.

In an embodiment, the fluid to be delivered to the treatment site contains one or more nutrients or therapeutic agents for promoting wound healing.

In a second aspect, the present invention provides a system for draining fluid from a treatment site or delivering fluid to a treatment site in the body of a patient. The system comprises the device described above in relation to the first aspect, a conduit releasably coupled to either the port of the device or to a fluid impermeable dressing, a reservoir located external to the body of the patient, the reservoir in fluid communication with the conduit for receiving fluid from the conduit or delivering fluid to the conduit, and a source of pressure coupled to the conduit for delivering positive pressure or negative pressure to the device.

In an embodiment, the source of pressure is capable of delivering negative pressure to the device so that fluid is drained from the treatment site into the device and transferred through the conduit to the reservoir. The pressure may be applied continuously, or vary. For example the pressure may be applied intermittently, pulsed, or altered over the course of treatment.

In an embodiment, the source of pressure is capable of delivering positive pressure to the device so that fluid in the reservoir is transferred through the conduit into the device and to the treatment site. The pressure may be applied continuously, or vary. For example the pressure may be applied intermittently, pulsed, or altered over the course of treatment.

In an embodiment, the treatment site is an exposed area of tissue, such as muscle or subcutaneous tissue, in an open surgical or tunnelled wound.

In a third aspect, the present invention provides a method of draining fluid from a treatment site or delivering fluid to a treatment site in the body of a patient. The method comprises implanting the device described above in relation to the first aspect at the treatment site, coupling a conduit to the port of the device, the conduit being connected to a reservoir located external to the body of the patient for receiving fluid from the conduit or delivering fluid to the conduit, and delivering negative pressure to the device so that fluid is drained from the treatment site into the device and transferred through the conduit to the reservoir, or delivering positive pressure to the device so that fluid in the reservoir is transferred through the conduit into the device and to the treatment site. Optionally, a wound dressing may be applied to an incision near the treatment site, and negative pressure applied to the wound dressing, the wound dressing negative pressure supply being also coupled to the positive or negative pressure source. In an embodiment, the treatment site may be an exposed area of tissue, such as muscle or subcutaneous tissue, in an open surgical or tunnelled wound.

### BRIEF DESCRIPTION OF THE FIGURES

FIG. 1 is a cross-sectional view of an abdominal space showing the placement of a prior art drain device for the management of a seroma adjacent to an abdominal wall or muscle.

FIGS. 2a to 2d show various treatment stages using a device according to one embodiment of the invention, where FIG. 2a shows the device implanted adjacent the seroma, FIG. 2b shows the seroma and accompanying dead space reduced in size, FIG. 2c shows the seroma fully drained and the source of negative pressure disconnected from the port of the device, and FIG. 2d shows the device fully resorbed.

FIGS. 3a to 3c show various configurations of impermeable dressings used to facilitate connection of an externally located port on embodiments of the device, to a vacuum or positive pressure source.

FIG. 4 is a schematic, conceptually showing an open abdomen and placement of a device of one embodiment of the invention on the abdominal wall muscle with the port of the device located on the exterior surface of the skin.

FIGS. 5a to 5d are partial section views showing various exemplary truss and sheet configurations used to create fluid flow channels, where FIG. 5a shows an embodiment having and arch-shaped truss with lower joining or bracing truss members provided on an underside of the lower flexible sheet, FIG. 5b shows a similar embodiment to FIG. 5a, but with the lower truss members sandwiched between two sheets, FIG. 5c shows an embodiment with upper and lower sheets held apart by an arch-type truss, and FIG. 5d shows a similar embodiment to FIG. 5c, but with the two sheets stitched together along the edges of the channel.

FIG. 6 is a cut-away perspective view illustrating one form of an arch-shaped truss creating a channel between two flexible sheets.

FIG. 7 is a cut-away perspective view illustrating an alternative arch-shaped truss with bracing truss members at the sides and apex of the truss.

FIGS. 8a and 8b show an embodiment with apertures provided through the top flexible sheet to permit fluid exchange through the surface to channels of the device, where FIG. 8a is a cut-away perspective view revealing the truss structure, and FIG. 8b is a perspective view better illustrating the apertures.

FIG. 9 is a cut-away perspective view showing an embodiment with apertures provided through upper and lower flexible sheets to permit fluid exchange across both surfaces of the device.

FIG. 10 is a cut-away perspective view showing a further alternative truss structure having and arch-type truss portion and diagonal bracing truss members provided across the base of the truss.

FIGS. 11a to 11d are end views of channels showing various alternative device truss members, where FIG. 11a shows an embodiment having an arch-shaped truss as also shown in FIG. 6, FIG. 11b shows an embodiment with a single arch-shaped truss forming a channel between two polymeric layers, FIG. 11c shows an embodiment with a corrugated truss structure, a plurality of sub-channels being formed between corrugations, and FIG. 11d shows an embodiment where the truss comprises three spaced apart elongate truss members that form a channel by virtue of their diameter.

FIGS. 12a and 12b show an embodiment device of the invention having multiple channels extending from a hub, between upper and lower flexible sheets, where FIG. 12a is a top perspective view, and FIG. 12b is an underside perspective view.

FIGS. 13a and 13b show an embodiment similar to the one in FIGS. 12a and 12b, but where one surface of the device ends at the exterior surface of the skin for positioning the port externally where FIG. 13a is a top perspective view, and FIG. 13b is an underside perspective view.

FIGS. 14a to 14d are perspective views showing various alternative embodiments of a device with multiple channels extending from a hub, with a portion of the upper sheet cut-away to reveal the respective truss structures, where FIG. 14a shows one embodiment device with no apertures in the channel wall and no web apertures, FIG. 14b shows the device of FIG. 14a but including web apertures to allow tissue contact between adjacent channels, FIG. 14c shows the embodiment of FIG. 14b but further including apertures in the top sheet at the channel walls for fluid passage into the channels; and FIG. 14d shows the device of FIG. 14a but including apertures in the top sheet at the channel wall for fluid passage into the channels.

FIGS. 15a and 15b show a similar embodiment device to that shown in FIG. 14d, but additionally including apertures in the lower sheet at the channel walls for fluid passage into the channels, where FIG. 15a is a top perspective view, and FIG. 15b is an underside perspective view.

FIGS. 16a and 16b show a similar embodiment device to that shown in FIG. 14d, but in which the channel walls of the main channel extend directly from the port of the device do not include apertures through the walls, where FIG. 16a is a top perspective view, and FIG. 16b is an underside perspective view.

FIGS. 17a and 17b show a similar embodiment device to that shown in FIG. 14a, but including apertures in the lower sheet at the channel walls for fluid passage into the channels, where FIG. 17a is a top perspective view, and FIG. 17b is an underside perspective view.

FIGS. 18a and 18b show a similar embodiment device to that shown in FIGS. 14a and 14b, but where one surface of the device terminates at the surface of the skin.

FIG. 19 is a cut-away perspective view showing yet a further embodiment channel, in which the truss is substantially helical and positioned between two flexible sheets.

FIG. 20 is a cut-away perspective view showing a similar embodiment to FIG. 19, but further including stitching along sides of the channel to join the two flexible sheets.

FIG. 21 is a cut-away perspective view showing the channel structure of FIG. 20, with the truss extending into a coupling tube for connecting to the supply conduit to provide of positive of negative pressure to the device.

FIG. 22 is a cut-away perspective view showing the channel structure of FIG. 20, with the truss extending into a releasably connected conduit for the supply of positive of negative pressure to the device.

FIG. 23 is a cut-away perspective view showing yet a further embodiment channel having a substantially helical truss member with side bracing members, and the channel walls formed by a single flexible sheet with its edges stitched together at a side seam.

FIG. 24 is a cut-away perspective view of an embodiment similar to that in FIG. 23, but with a single row of apertures provided in the channel walls.

FIG. 25 is a cut-away perspective view of an embodiment similar to those in FIGS. 23 and 24, but with multiple rows of apertures provided in the channel walls.

FIG. 26 is a cut-away perspective view showing a further embodiment channel in which the truss has two overlapping helical members and side bracing members, and the channel walls formed by a single flexible sheet with edges adhered together at a side seam.

FIGS. 27a and 27b are cut-away perspective views showing embodiments having the channel structure of FIG. 25, where FIG. 27a shows a device in which the truss structure extends into an enlarged coupling conduit for the supply of positive or negative pressure to the device, and FIG. 27b shows a device where the truss structure is modified adjacent the port of the device to accommodate coupling to a conduit.

FIGS. 28a and 28b are partial perspective views of a device in which the outer layer is secured by an absorbable locking component having tissue retention barbs, where FIG. 28a is a right side perspective view and FIG. 28b is a left side perspective view.

FIG. 29 is an illustrative schematic view showing the placement of one embodiment single channel device at a treatment site and having an internally positioned port connected to a source of negative or positive pressure.

FIGS. 30a and 30b are views corresponding to FIG. 29 but showing concurrent treatment of an incision wound using negative pressure wound therapy; where FIG. 30a shows a treatment area similar to that in FIG. 29, and FIG. 30b shows treatment of a treatment area that extends to adjacent the incision wound.

FIG. 31 is a view corresponding to FIG. 29 but additionally showing one end of the device connected to a supply of treatment fluid.

FIG. 32 is a view corresponding to FIG. 31 but with the supply of treatment fluid and the source of negative or positive pressure coupled to the device via a single implanted port.

FIGS. 33a to 33d illustrate one method of manufacturing a truss having two helical members and two side bracing members, where FIG. 33a shows a first truss member wound around a central mandrel, FIG. 33b shows two elongate side bracing members being bonded to the first truss member, FIG. 33c shows a second truss member wound around the central mandrel, the first truss member and the side members and bonded to the first truss member and side members, and FIG. 33d shows the central mandrel being removed from the truss.

FIGS. 34a and 34b illustrate an alternative method of manufacturing a truss, where FIG. 34a shows a first truss member wound around a central mandrel and two elongate side bracing members, and FIG. 34b shows the central mandrel being removed from the truss.

FIGS. 35a and 35b are cross-sectional views of one embodiment of the device implanted at a treatment site, where FIG. 35a shows the device as initially implanted in an area of dead space within a subcutaneous tissue space; and FIG. 35b shows the reduction of dead space after a treatment period.

FIGS. 36a and 36b are views corresponding to FIGS. 35a and 35b, with the device additionally connected to a topically applied wound treatment device to simultaneously treat an incisional wound, where FIG. 36a shows the device as initially implanted in an area of dead space within a subcutaneous tissue space and the dressing as initially applied; and FIG. 36b shows the reduction of dead space after a treatment period.

FIGS. 37a and 37b are illustrative embodiments of a single channel device configured to permit alternative fluid flow paths to form in the event of a blockage occurring, where FIG. 37a shows fluid flow in an unblocked device, and FIG. 37b shows the change in fluid flow in response to a blockage.

FIGS. 38a and 38b are illustrative embodiments of the device of FIGS. 37a and 37b, but including connecting webs or sleeves to assist to maintain the shape of the device, where FIG. 38a shows the overall device, and FIG. 37b is an enlargement of a portion of the device near the port.

FIGS. 39a and 39b are perspective views showing a further embodiment truss and channel releasably coupled to a two lumen conduit, with the truss structure extending into the conduit, where FIG. 39a shows the conduit as a transparent member, and FIG. 39b is a cut-away perspective view.

FIGS. 40a to 40c, are top perspective views of the embodiment in FIGS. 39a and 39b, showing the sequence of removing the releasably coupled conduit from the truss, where FIG. 40a shows the conduit coupled to the truss and channel, FIG. 40b shows the conduit in the process of being removed from the truss, and FIG. 40c shows the conduit removed from the truss.

FIG. 41 is a cross-sectional view of one embodiment of the device implanted at an open treatment site where the remaining wound has been covered with a dressing to facilitate treatment.

FIG. 42 is a cross-sectional view of one embodiment of the device implanted at an open treatment site, the device is shown with a topically applied wound treatment placed on top of the implant.

### DETAILED DESCRIPTION

### I. Definitions

The term “bioresorbable” as used herein means able to be broken down and absorbed or remodelled by the body, and therefore does not need to be removed manually.

The term “treatment site” as used herein refers to a site in a human or animal body where surfaces of muscle tissue, connective tissue or skin tissue have been separated during surgery or as a result of trauma or removal.

The term “propria-submucosa” as used herein refers to the tissue structure formed by the blending of the lamina propria and submucosa in the forestomach of a ruminant.

The term “lamina propria” as used herein refers to the luminal portion of the propria-submucosa, which includes a dense layer of extracellular matrix.

The term “extracellular matrix” (ECM) as used herein refers to animal or human tissue that has been decellularised and provides a matrix for structural integrity and a framework for carrying other materials.

The term “decellularised” as used herein refers to the removal of cells and their related debris from a portion of a tissue or organ, for example, from ECM.

The term “polymeric material” as used herein refers to large molecules or macromolecules comprising many repeated subunits, and may be natural materials including, but not limited to, polypeptides and proteins (e.g. collagen), polysaccharides (e.g. alginate) and other biopolymers such as glycoproteins, or may be synthetic materials including, but not limited to polyglycolic acid, polylactic acid, P4HB (Poly-4-hydroxybutyrate), polylactic and polyglycolic acid copolymers, polycaprolactone and polydioxanone.

### II. Device

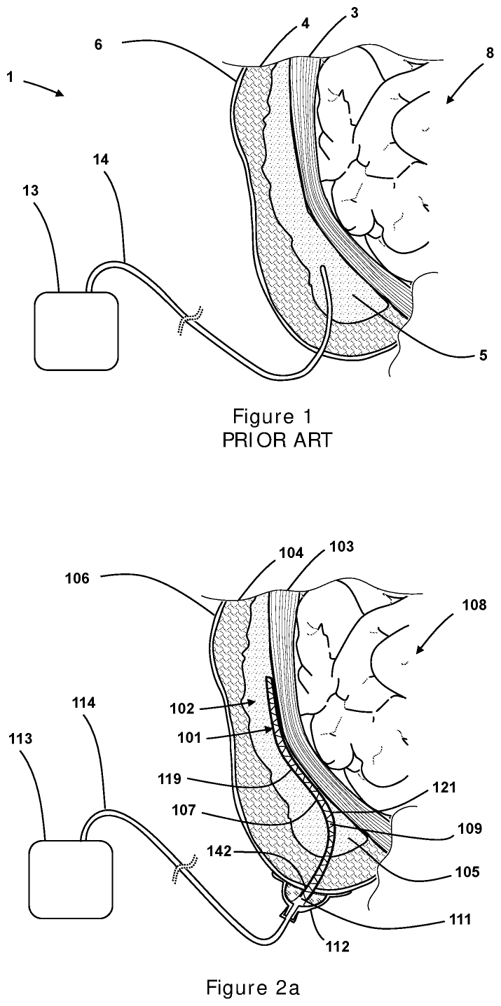

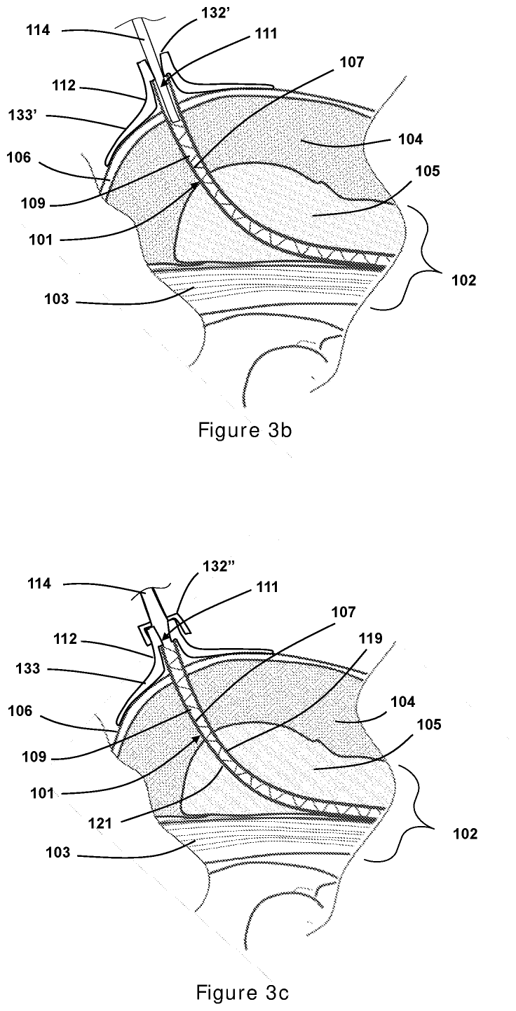

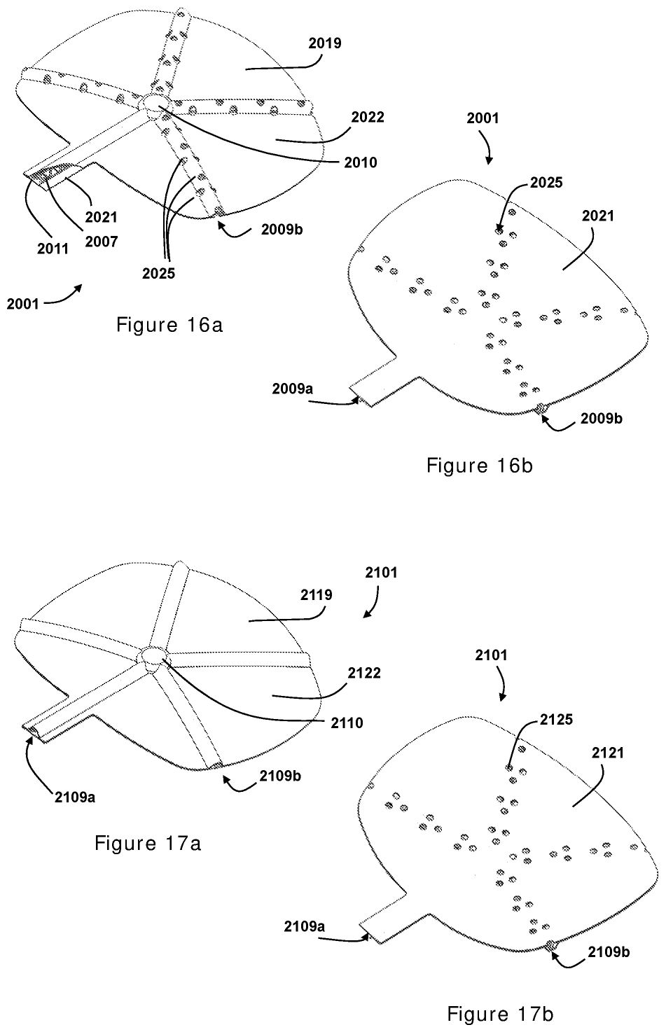

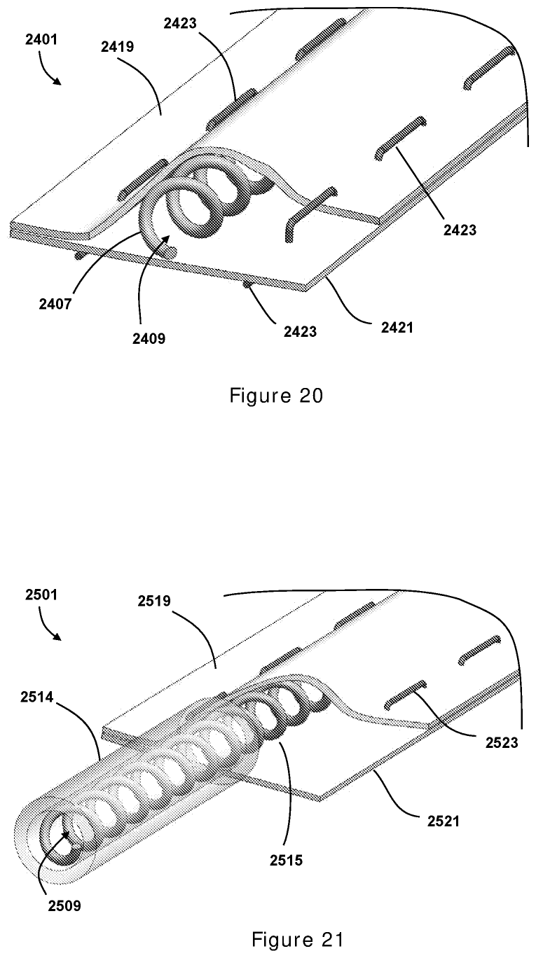

Various embodiments of the device and system of the present invention will now be described with reference to FIGS. 1 to 42. In these figures, like reference numbers are used to indicate like features. Where various embodiments are illustrated, like reference numbers may be used for like or similar features in subsequent embodiments but with the addition of a multiple of 100, for example 2, 102, 202, 302 etc. Directional terminology used in the following description is for ease of description and reference only, it is not intended to be limiting. For example, the terms ‘front’, ‘rear’, ‘upper’, ‘lower’, and other related terms are generally used with reference to the way the device is illustrated in the drawings.

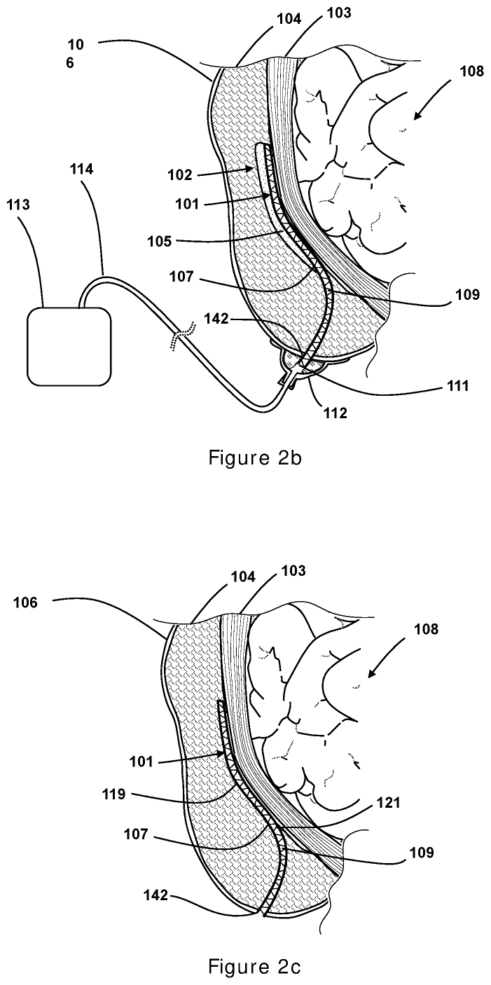

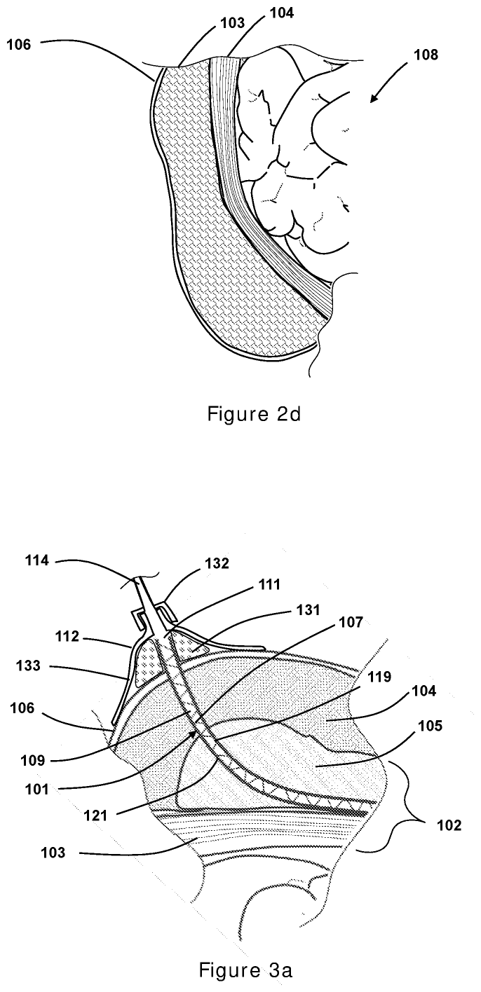

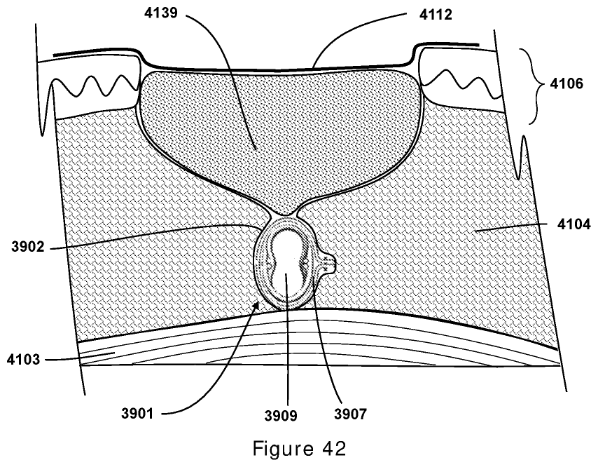

FIGS. 2a to 28b, 37a to 38b, and 39a to 40c show embodiments of a bioresorbable device 101 for implantation at a treatment site 102 in the body of a patient, for the purpose of draining fluid from the treatment site or delivering fluid to the treatment site. The treatment site 102 may be a space between surfaces of muscle tissue 103, connective tissue 104 or skin tissue that have been separated during surgery or as a result of trauma. The treatment site may be the site of a seroma 105 or hem atom a, or maybe used as a prophylactic following surgical excision of tissue. Alternatively, the treatment site may be an open wound such as following trauma, injury or surgical excision of necrotic or infected tissue (FIGS. 41 and 42).

The device 101 has a bioresorbable resilient truss 107 that, in use, holds two tissue surfaces 103, 104 spaced apart, thereby defining a channel 109 into which fluid from the treatment site can drain or from which fluid can be delivered to the treatment site. A port 111 in the form or an opening at one end of the truss 107 is in fluid communication with the channel 109 and allows for connection of the channel with a source of negative pressure or positive pressure 113. The two tissue surfaces 103, 104 need to be held apart because they would otherwise collapse together, particularly under application of negative or reduced pressure (vacuum) to assist with fluid drainage.

In some alternative embodiments the device 101 could be operably connected to one or more other devices, implanted at different respective sites for treating the respective sites with the same pressure source.

In some alternative embodiments the device could be in contact with another wound treatment device also connected to a source of negative or positive pressure.

FIGS. 5 to 11d, 19 to 28b and 39a to 40c illustrate various exemplary embodiments of the resilient truss 107. The truss 107, 207, 307, etc. may define a single channel 109, 209, 309, etc. or a plurality of interconnected channels, for example in a branched structure. The truss 107, 207, 307, etc. is flexible in its longitudinal direction to allow the channel(s) to flex to substantially conform to the contours of the treatment site 102 and to reduce or prevent localised irritation or abrasion to the surrounding tissue. The truss is a three-dimensional structure with sufficient strength to hold the two tissue surfaces 103, 104 apart, at least at the time of implantation, without the truss buckling or the channel collapsing or kinking under movement or application of clinically appropriate levels of negative pressure. If the two tissue surfaces 103, 104 were to collapse together, fluid flow would be severely restricted and possibly blocked altogether.

As well as having sufficient cross-sectional strength to hold the tissue surfaces apart, the truss 107 is also resilient in its radial directions. This resilience allows some flexing of the channel walls under force to prevent or reduce damage to the tissue but ensures that the channel 109 will return to its original configuration when the force is removed. For example, if tissue movement results in increased pressure on the truss.

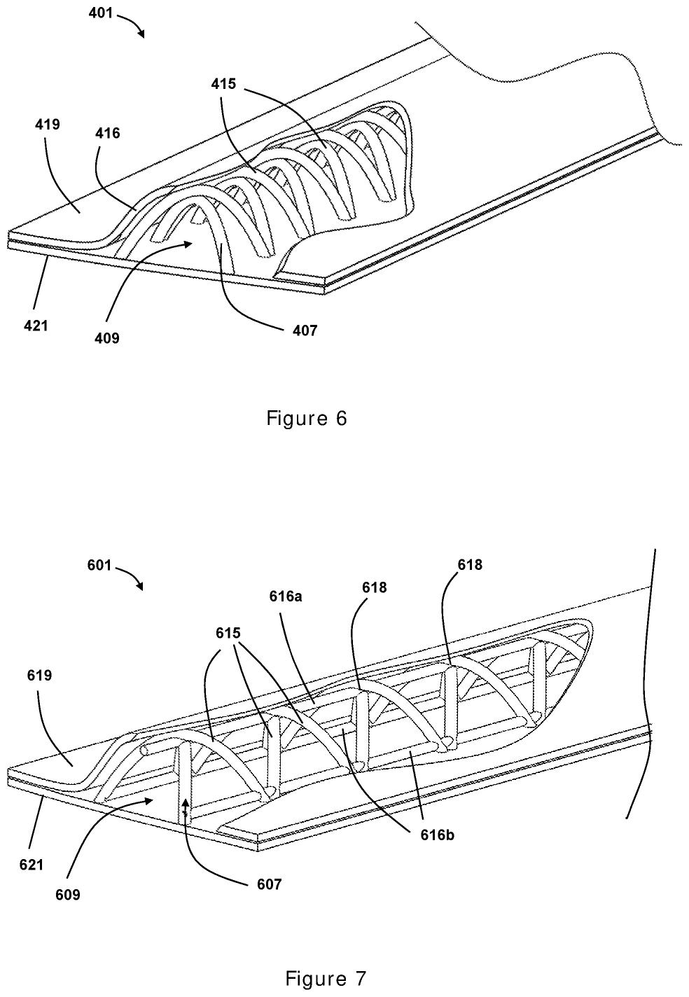

With reference to FIGS. 6 and 19 as examples, the truss 407, 2307 comprises at least one flexible elongate truss member 415, 2315 arranged to form a framework for the channel 409, 2309. The elongate truss member(s) 415 preferably has an arc length longer than the length of the channel 2309 or portion of the channel 409 along which it extends. Preferably the truss member 2315 or at least one truss member 415 is curved so as to follow a curved contour of the internal surface of the channel wall 417, 3217. For example, the truss member(s) 415, 2315 may be arcuate, helical, sinuous, or otherwise curved, substantially following the curvature of the channel wall. The truss may additionally or alternatively comprise substantially straight truss members. The truss member(s) may comprise a filament/tread.

The truss may have an ‘open’ form, where the truss member(s) lie only or predominantly along upper or lower and/or side portion of the channel, for example forming an arch-shaped truss 407, 507, 607, 707, 807 as shown in FIGS. 5c, 5d, and 6-10, with the respective channel 409, 509, 609, 709, 809 being defined under the arch. With reference to FIG. 6, curved truss members 415 are arranged to cross pathways with each other in a manner such that the underlying truss member assists to prevent or resist the collapse of the overlying member when compressed. With reference to FIG. 7, the truss 607 may further comprise bracing truss members 616a, 616b bonded or otherwise joined to the curved truss members 615 at discrete points 618, to hold the respective bonded or joined points of the truss members 616 in spaced apart relation, thus reducing or preventing collapse of the channel walls 617 due to relative movement of respective truss member portions. For example, in the exemplary devices 601, 701, 801, 901 of FIGS. 7 to 9, the arched shaped truss comprises two elongate side bracing members 616b, 716b, 816b and an elongate bracing member 616a, 716a, 816a at the apex of the arch. The bracing members 616a, 616b, 716a, 716b, 816a, 816b have a length substantially the same as the length of the channel or the portion of the channel along which they extend.

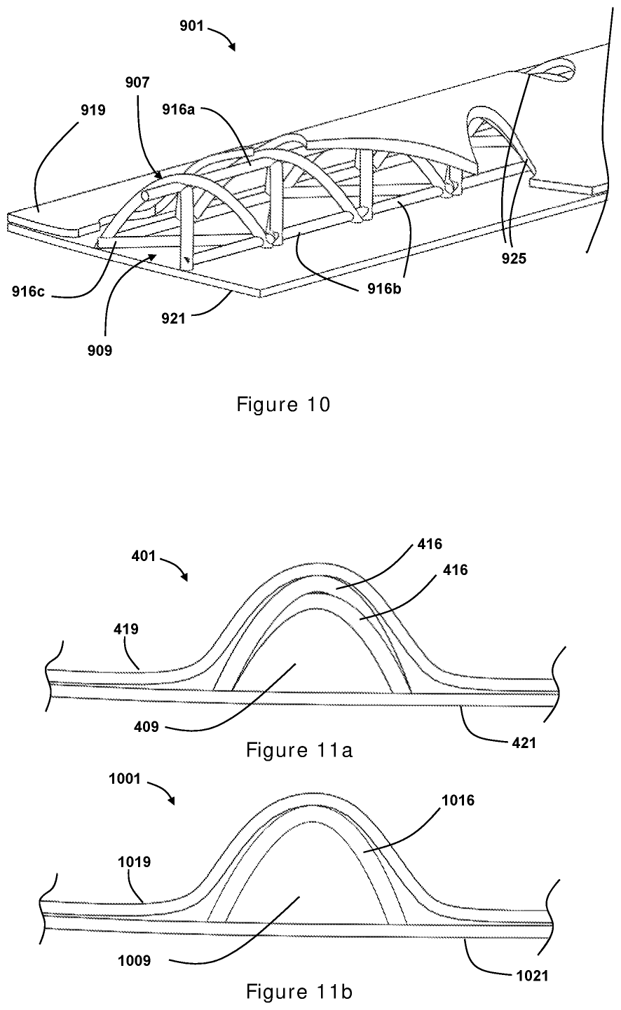

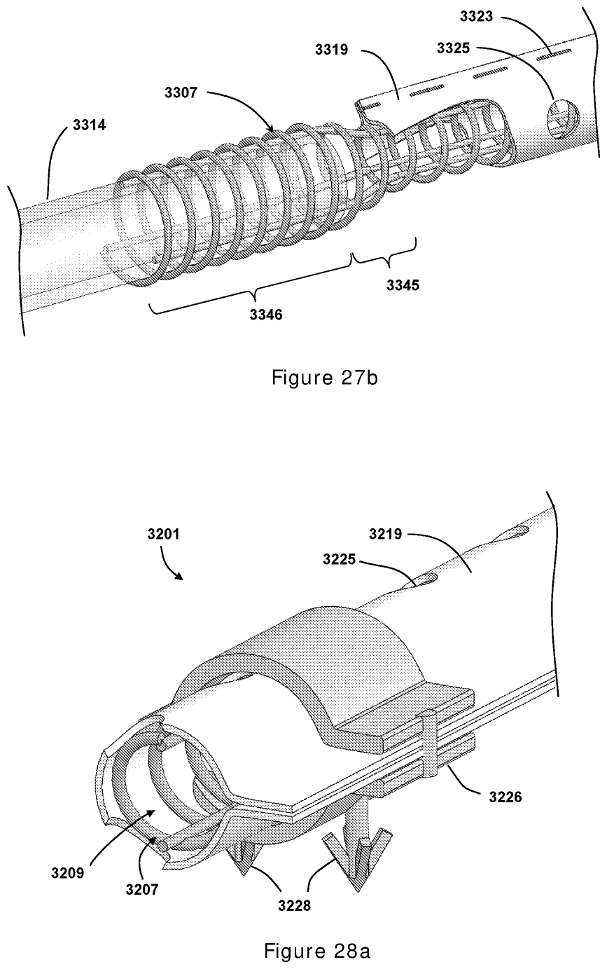

Alternatively the truss may have a ‘closed’ form, in which the truss is tubular in nature, providing support to the tissue surfaces in all radial directions. For example, the embodiment of FIG. 10 additionally comprises a series of diagonal bracing struts 916c along the base of the arch to substantially maintain the spacing between the lower edges of the arch 916b. FIGS. 19 to 26 further illustrate an exemplary embodiments having a closed truss form 2307 that includes at least one substantially helical truss member 2315 defining a cylindrical channel 2309. As shown in FIGS. 23 to 28b, the helical truss 2707 may further comprise one or more bracing truss members 2716 bonded or otherwise joined to the helical truss member 2715 at discrete points, to hold adjacent winds of the helical truss member 2715 spaced apart, thus reducing or preventing collapse of the tube due to relative movement of adjacent winds of the truss member. The embodiments shown in FIGS. 23 to 28b, comprise two elongate side bracing members 2716 that have a length substantially the same as the length of the channel or the portion of the channel along which they extend. Embodiments may optionally include additional bracing members, for example three bracing members (as shown in FIG. 27b), or alternatively have fewer bracing members.

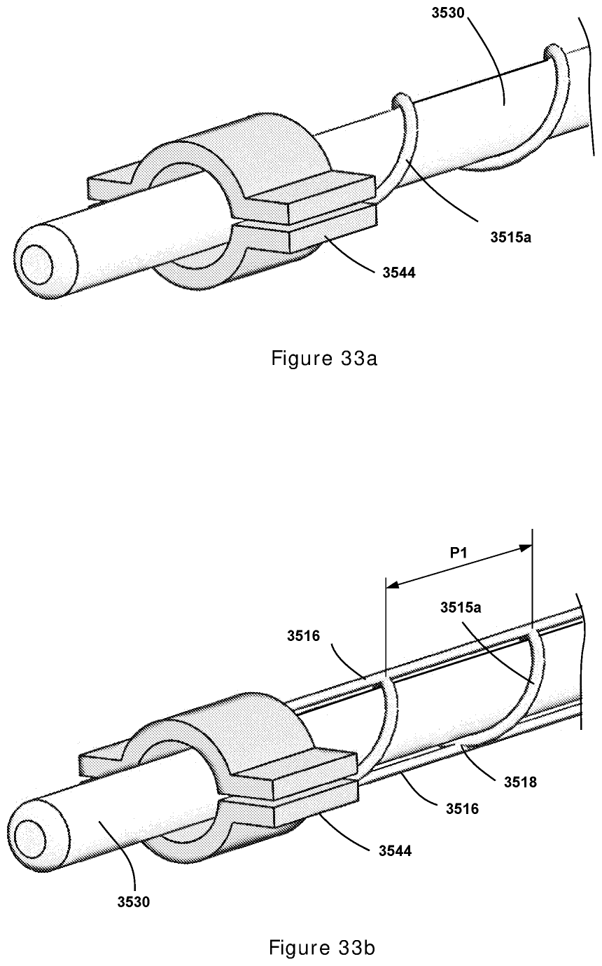

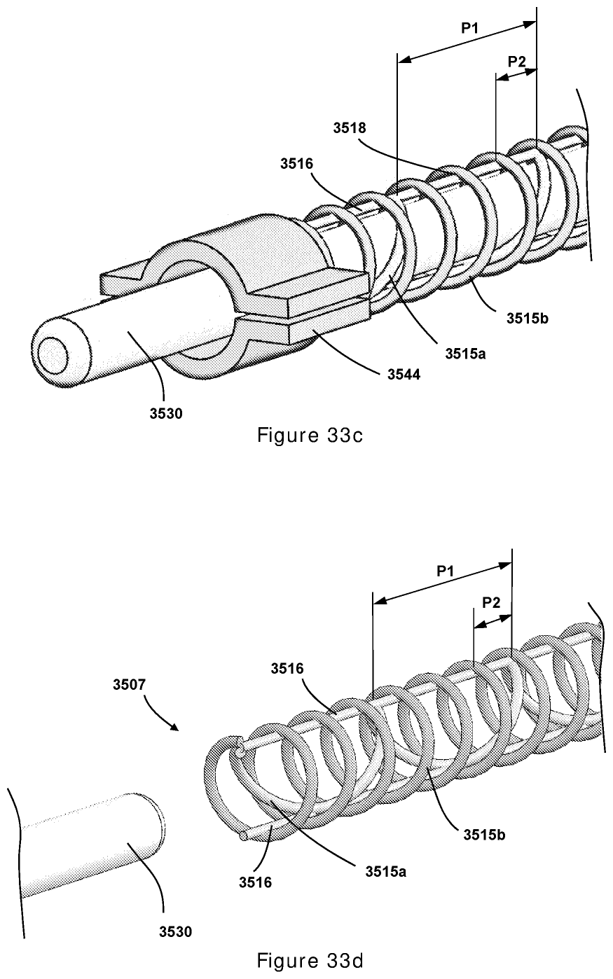

The truss may comprise a plurality of helical truss members. For example, FIG. 33d illustrates a further embodiment truss 3507 comprising overlapping first and second substantially helical truss members 3515a, 3515b. The first truss member 3515a has a first pitch length P1, and the second truss member 3515b has a second pitch length P2 that is greater than the first pitch length P1. In the embodiment shown, the second pitch length P2 is about 3.5 times the first pitch length P1. However, other ratios are anticipated, for example the first pitch length P1 may be about 4.5 or between about three to about five times the second pitch length P2, or between about two to about ten times the first pitch length P1.

In the embodiment shown, the truss 3507 further comprises two elongate side bracing members joined to both the first and second helical truss members. The elongate side members have a length substantially the same as the length of the channel or the portion of the channel along which they extend. In alternative embodiments, the truss may have more or fewer bracing members, and/or may have more than two helical members.

The first and second truss members 3515a, 3515b are bonded together and/or bonded to the bracing members 3516 at discrete points 3518 where the members overlap each other. This exemplary structure having multiple helical members bonded together advantageously allows a higher strength truss to be created using less truss material.

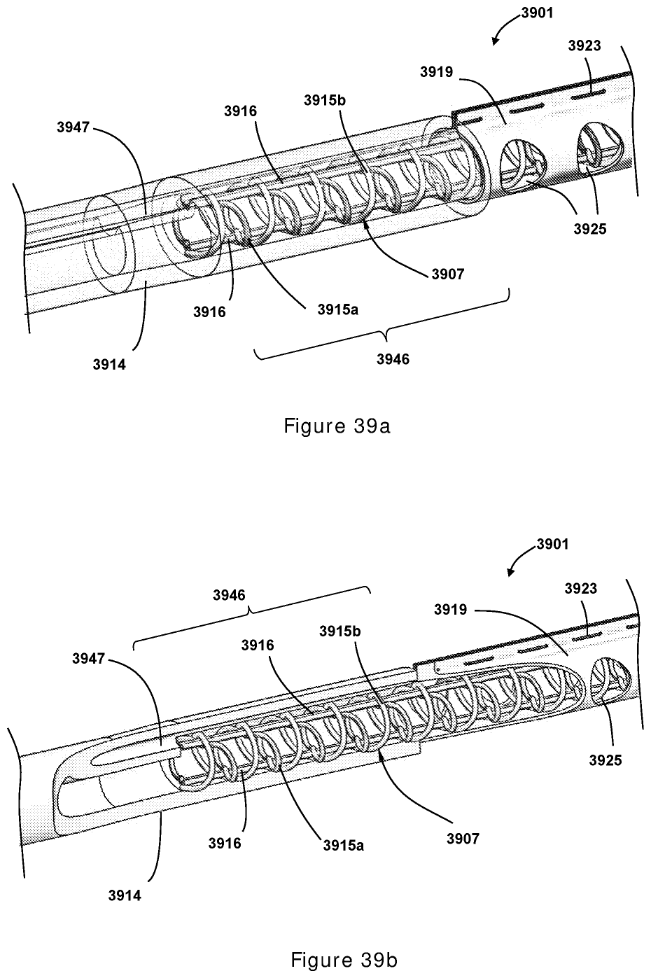

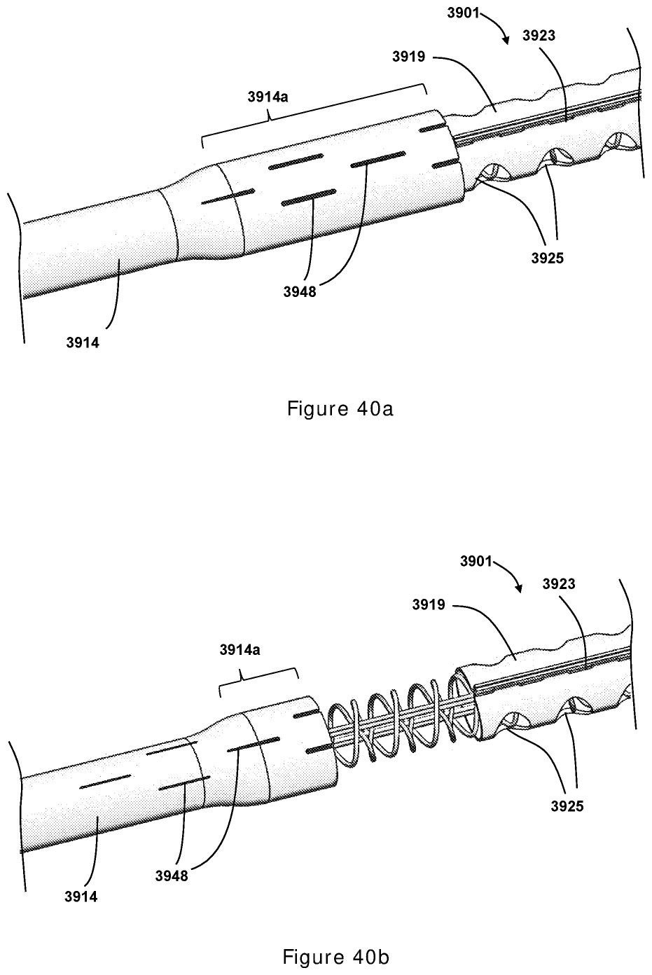

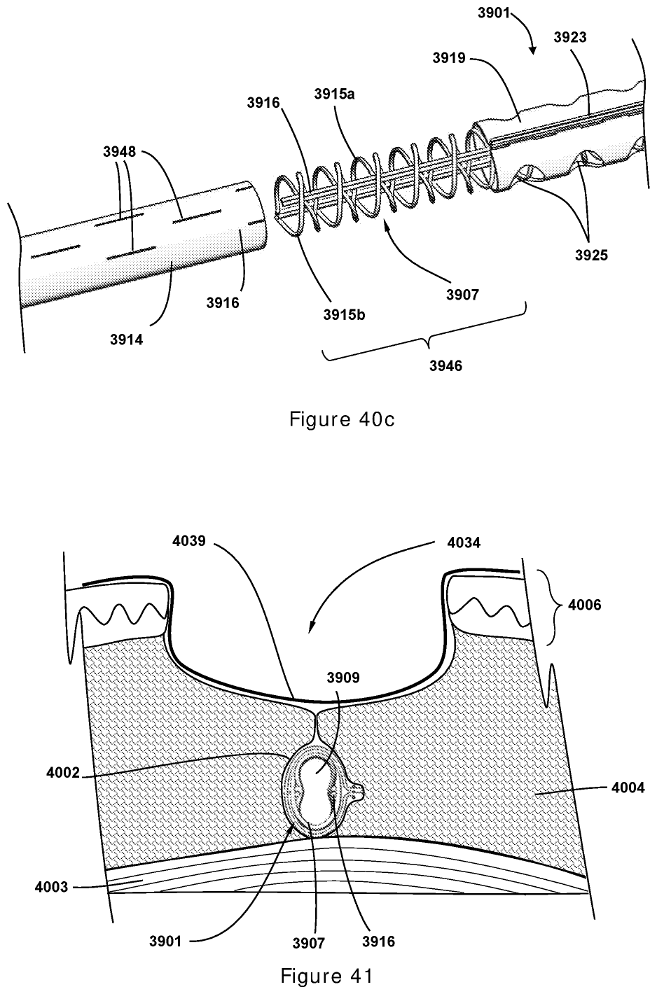

FIGS. 39a to 40c show a further embodiment truss 3907 having first and second substantially helical truss members 3915a, 3915b with equal pitch lengths but winding in opposing directions. The first and second helical truss members 3915a, 3915b define a channel 3909 with a non-circular cross-sectional profile. In the embodiment shown (see FIGS. 41 and 42), the channel 3909 has an oval or elliptical cross sectional profile with a major dimension and a minor dimension that is less than the major dimension. When placed in a wound between two tissue surfaces, the device is preferably orientated with the major axis lying along the interface of the two tissues, such that the spacing between the two tissue surfaces corresponds to the minor dimension. This allows the two tissue surfaces to be closer together to better facilitate healing than in an embodiment with a cylindrical truss of the same cross sectional area, while also improving patient comfort.

The truss comprises four elongate bracing truss members 3916, two at a top of the truss and two at a bottom of the truss as viewed in FIGS. 40a and 40b (on the minor axis), to hold adjacent winds of the helical truss members 3915a, 3915b spaced apart. The use of pairs of bracing members 3916 provides additional support and resistance to crushing or kinking of the truss 3907 compared to an embodiment with two single bracing members. However, in alternative embodiments, the truss may comprise a single top bracing member and a single lower bracing members, and these bracing members may be thicker and/or wider than the helical truss members, for example in the form of a tape, to provide improved bracing. Each bracing truss member 3916 lies between the two truss members 3915a, 3915b—with the first truss member 3915a being bonded or otherwise joined to an inner surface of the bracing truss members 3916 at discrete points, and the second truss member 3915b being bonded or otherwise joined to an outer surface of the bracing truss members 3916 at discrete points. The first and second truss members 3915a, 3915b overlap each other at the point where they are bonded to the bracing truss members. As best illustrated in the cross-sectional in use view of FIGS. 41 and 42, the first truss member 3915a has sections that kink inwards where the first helical truss member 3915a is joined to respective bracing truss members 3916, to accommodate the bracing truss members 3916 and the second truss member 3915b. This inward kinking of the first truss member 3915a around the bracing members 3916 helps to reduce the likelihood the channel 3909 will be completely blocked should the truss be squashed. The first truss member 3915a and bracing members limit movement of the side walls 3919 towards each other, allowing some flow, particularly on either side of the bracing members, when opposite bracing members are pressed towards each other.

This exemplary structure with non-circular cross sectional profile and bracing members on the minor axis, advantageously allows for the truss 3907 to have more flexibility in one direction while also preventing kinking or collapsing of the truss in the sections between the helical truss members 3915a, 3915b.

For both open and closed form trusses, the number and nature of any bracing members will depend on the strength characteristics of the constituent truss members, the number of truss members, their configuration, and the cross-sectional area of the channel, a truss having an open form may comprise one or more elongate truss members to brace the other truss members.

In some preferred embodiments of the invention, the channel has a cross sectional area of about 28 mm². This may be provided by a cylindrical channel with diameter or maximum width of about 6 mm, or alternatively by an oval or elliptical channel. However, a range of cross sectional areas are possible, and different applications may require channels of different cross sections. For example, in alternative embodiments, the channel may have a cross sectional area in the range of about 3 mm² to about 80 mm², preferably about 12 mm² to 50 mm², i.e. in a cylindrical channel embodiment, a diameter or width in the range from approximately 1 mm to 10 mm, preferably about 4 mm to about 8 mm. The cross-sectional area may be constant or may vary. The larger cross-sectional area channel compared to conventional fluid drainage devices provides a more favourable, lower pressure drop over the length of the channel and is also favourable for preventing blockages.

The resilient truss also provides a more effective structure to provide a channel between two surfaces by reducing the overall mass of the synthetic material per unit length when compared to the existing prior art devices.

The truss also has a porous structure which permits free fluid exchange from the internal channel to the surrounding area for more effective passage of fluid when compared to the closed form nature of the existing prior art which is dependent on small diameter apertures/perforations to pass fluid into the channel. Synthetic bioresorbable polymers also typically release acid when they breakdown which can cause elevated levels of inflammation where existing prior art devices persist for longer given the thickness of the sections.

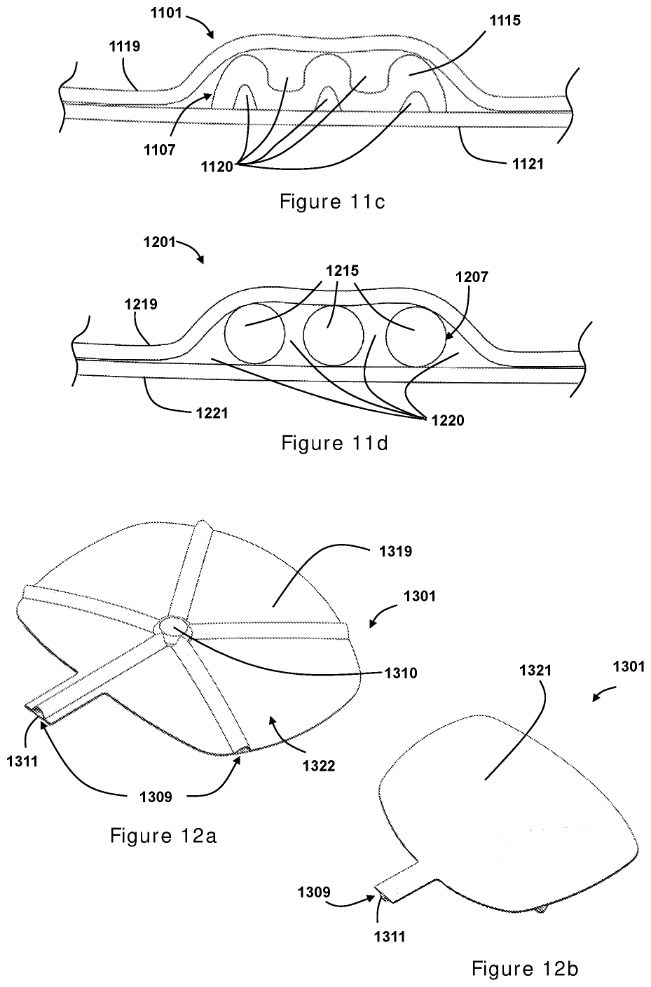

As a further alternative illustrated in FIGS. 11c and 11d, the truss 1107, 1207 may comprise lengths of tape of thread 1215, or corrugations 1115 with a thickness t corresponding to the desired thickness of the channel 1109, 1209. Sub-channels 1120, 1220 then form longitudinally along either side of the length of thread or tape 1215, between two lengths of thread or tape 1215, or form longitudinally within a cavity defined by the corrugations or other three-dimensional structure of the truss member 1115.

In some embodiments the truss 107 is implantable directly at the treatment site, such that the truss directly contacts surfaces of the treatment site. The surface of the treatment site would be formed from tissue (e.g. muscle tissue, connective tissue or skin) or bone of possibly a combination of tissue and bone. A wall or walls defining the channel is then formed by the tissue surfaces themselves, where they are held apart by the truss. The channels may be formed between the surface of one sheet of a flexible material and a surface of the treatment site.

Referring to FIGS. 5a to 28b, alternatively the device may comprise one or more flexible sheets, 219, 221 of a bioresorbable material, forming at least a portion of the channel wall 217. In some embodiments, the flexible sheet or sheets 219, 221 may only partly form the channel wall 217, with the remaining part of the channel wall formed by the tissue surface. That is, the channel may be formed between a surface of a flexible sheet and a surface of tissue or bone at the treatment site. Alternatively, the flexible sheet or sheets may form a major part or substantially the whole of the channel wall. Such an embodiment may either comprise two or more bioresorbable flexible sheets with the truss holding the sheets apart such that one or more channels are defined between facing surfaces of the sheets 219, 319, 419, etc, and 221, 321, 421, etc (see FIGS. 1 to 22), or a single flexible bioresorbable sheet 2719, 2819, 2919, 3019, etc may be wrapped around the truss to form the wall of the channel (see FIGS. 23 to 28b).

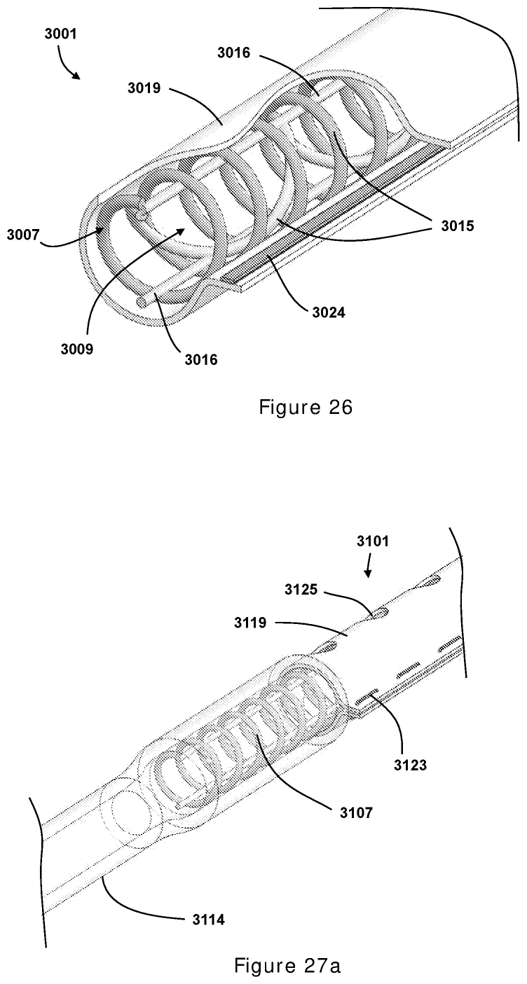

To secure the flexible sheet or sheets over or around the truss, the sheet or sheets may be stitched together along a seam at a side of the channel. FIGS. 5d and 20 to 22 illustrate exemplary embodiments in which two the upper and lower flexible sheets 519, 521 are stitched together along two side seams 523, 2423, 2523, 2623. In embodiments with a single flexible sheet wrapped around the truss, only a single side seam 2723, 2823, 2923 is necessary, as illustrated in FIGS. 23 to 25, 27a and 27b and 39a to 40c. Alternatively, rather than stitching, an adhesive 3024 may be used at the seam or seams to join together opposing sheet edges, for example, as illustrated in FIG. 26.

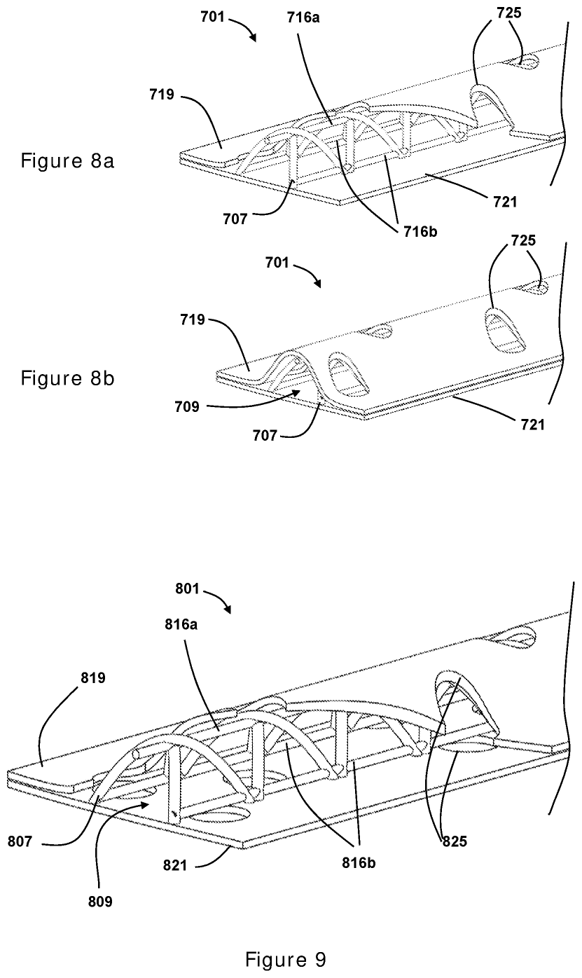

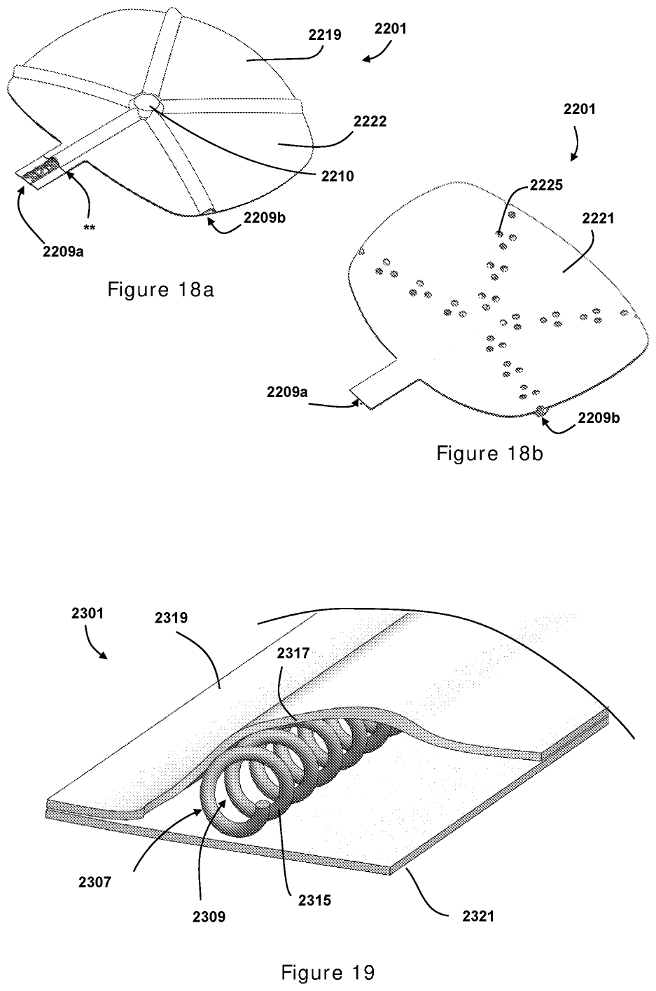

Referring now to FIGS. 8a to 9, a plurality of apertures 725, 825 may be provided in one or both of the flexible sheets 719, 819, 821, to facilitate fluid flow into the channel 709, 809. Apertures may be provided along one or more of: a top surface, side surface, and/or lower surface. The apertures 725, 825 may be aligned in one or more rows or may be staggered or otherwise arranged. FIGS. 8a, 8b, and 10 illustrate embodiments comprising two flexible sheets, with apertures 725, 925 provided only in the top sheet 719, 919 along the channel wall to facilitate fluid flow into or out of the channel. Whereas, FIGS. 9 and 14c to 16b illustrate alternative embodiments in which apertures 825, 1425, etc are provided in both the upper and lower sheets 819, 821, 1419, 1421, etc. along the channels to further improve fluid flow into or out of the channel. Alternatively, apertures 2125, 2225 may be only provided in the lower sheet 2121, 2221 along an underside of the channel walls, as illustrated in FIGS. 17a to 18b. The position of apertures on the channel walls may vary depended on the desired performance characteristics. For example, to exclude fluid delivery or extraction from a certain potion of the treatment area, apertures may be excluded from the channel walls in a corresponding portion of the device. Whereas channel wall apertures may be provided only along specific portions of the channel where the delivery of treatment fluid is desired to a particular region, for example, targeted pain relief to a particular surface such as a nerve or organ.

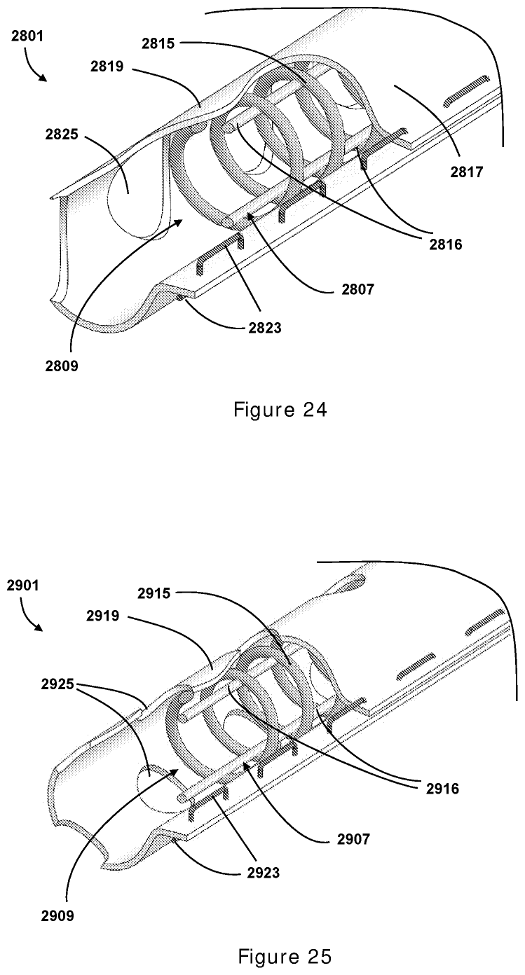

In embodiments with a single sheet wrapped around the truss, a plurality of apertures, for example arranged in one or more rows of apertures, may be provided in the flexible sheet. Where only a single row is provided, the apertures may be larger than for embodiments having two or more rows, to offer a similar rate of fluid flow into or out of the channel. For example, FIG. 24 illustrates a channel having a single row of apertures 2825, where the apertures 2825 are larger than those in the embodiment of FIG. 25, which has two rows of channel apertures. In the embodiment shown in FIG. 24 the surface area of the apertures 2825 is about 50% of the surface area of the channel wall 2717. However, it will be appreciated that in alternative embodiments, in portions of the channel wall comprising apertures, the surface area of the apertures may be from about 20% of the surface area of the channel wall 2717 to about 70%, preferably from about 30% to about 60%.

Because the device is bioresorbable and does not need to be removed, the size and spacing of the channel wall apertures 2825, 2925 is not limited by the need to limit trauma to tissue on removal. Existing removable drains which have apertures must balance the need for fluid transfer through the wall apertures of the device, with the need to reduce patient trauma during removal. Thus, existing drain devices limit the size of channel apertures to minimise the in-growth of tissue through the aperture, as in-growth is associated with increased trauma on removal of the device and contributes to blockages in the device, this reduction in the size of the apertures reduces the effectiveness of such devices in draining fluid. In contrast, in the present device, the truss underlying the apertures reduces tissue in-growth into the channel that may contribute to blockages while allowing the ingress or expulsion of fluid through gaps between adjacent portions of truss members.

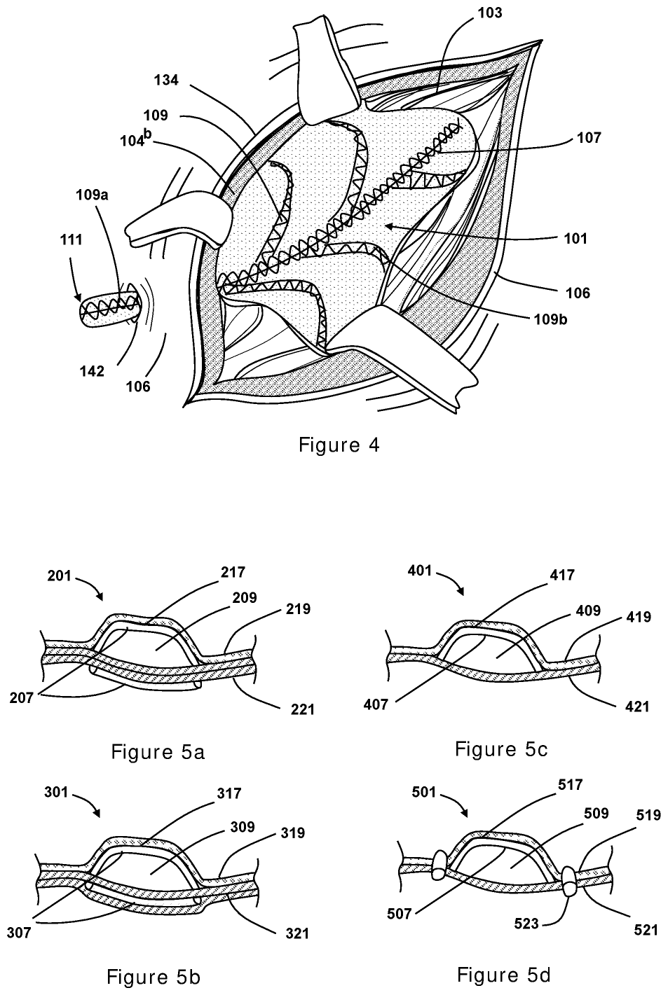

As mentioned above, the truss 103, 203, 303, etc. may define a single channel or a plurality of interconnected channels, for example as a branched structure. It will be appreciated that some devices of the invention will comprise many channels for fluid flow, for example 3, 4, 5, 6, 7, 8, 9, 10 or more channels, whereas some devices of the invention may comprise only 1 or 2 channels. FIGS. 4 and 12a to 18a illustrate various embodiment devices having a branched structure in which a plurality of secondary channels 109b, 1309b, 2209b branch off a primary channel 109a, 1309a, . . . , 2209a at one or more hubs or junctions 110, 1310, . . . , 2210. The secondary channels extend in different directions towards a periphery of the treatment site. The secondary channels may have a smaller cross sectional area than the primary channel, and/or one or more of the channels may taper along a portion of the channel.

The device may optionally include bioresorbable webbing 1322, . . . , 2222 between adjacent channels to maintain the relative positions of the channels and to improve the ease of implanting the device or assist. The webbing 1322, . . . , 2222 may be provided by one or both of the flexible sheets 1319, . . . , 2219 and 1321, . . . , 2221 as in the embodiments shown in FIGS. 12a to 18b.

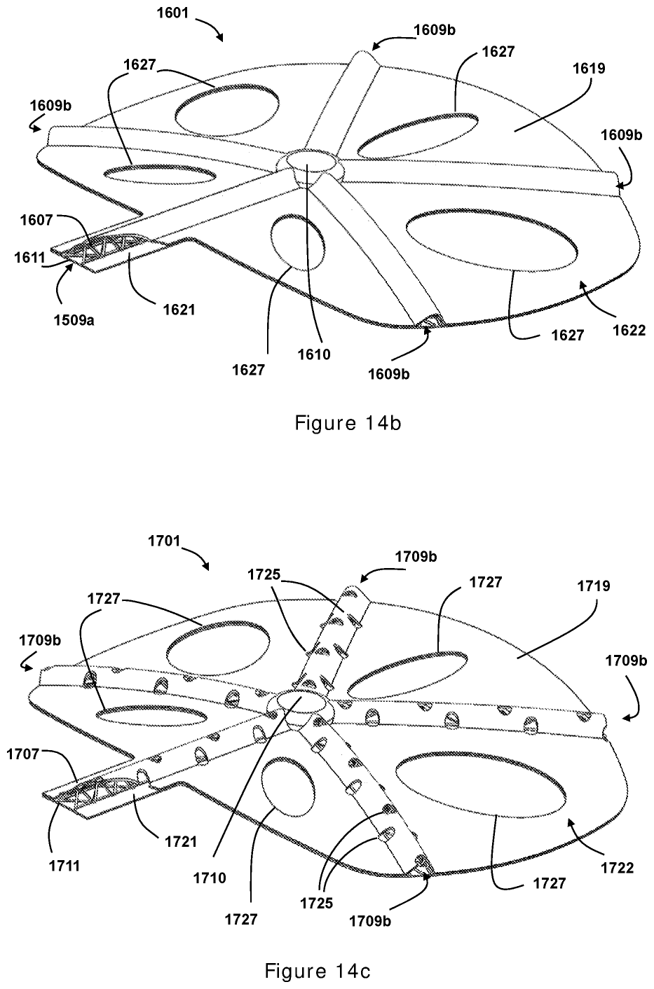

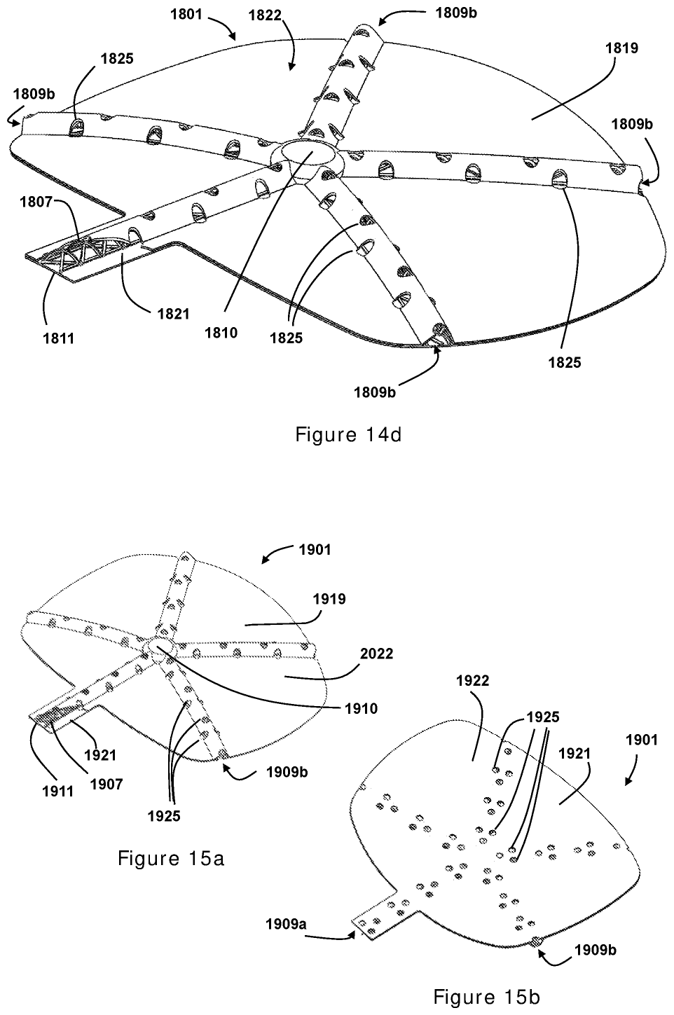

Generally, the inclusion of webbing undesirably increases the surface area of the device and which can create a barrier to the apposition of opposing tissue faces within a dead space therefore preventing the healing and subsequent reconnection of previously separated tissue. To minimise the physiological impact of the webbing, apertures 1627, 1727 may be provided in the webs (see FIGS. 14b and 14c). As well as reducing the surface area of the webs, these web apertures 1627, 1727 advantageously allow tissue-to-tissue contact, or tissue apposition, through the device for accelerated healing.

In alternative embodiments, the device may be a single channel device 3401, 3801. The single channel device 3401, 3801 may be elongate and flexible such that a surgeon can bend and configure the device 3401, 3801 as desired to fit within the treatment site 3402. For example, the device may be bent back and forward on itself, in a sinuous shape as illustrated in FIGS. 29 to 32 and FIGS. 37a to 38b, or formed into a coil or another suitable shape. Many potential configurations are possible in addition to the sinuous configuration shown, for example, a triangular configuration, e.g. for a mastectomy, an annular, elliptical or irregular shape, e.g. for a trauma site. The device may also be used in a linear configuration, particularly for positioning subcutaneously underneath an incision line such as in a caesarean incision, open abdominal wall repair, or in a T-shape configuration for a T-shape incision. A combination of shapes may be utilised, for example, by coupling a plurality of devices together to treat multiple sites within the body.

The single line shape also could work well within a minimally invasive surgical procedure such as a laparoscopic where it may be deployed following surgery as a prophylactic or retrospectively to treat a seroma or as a means to cyclically instil drugs to treat infections or diseases etc.

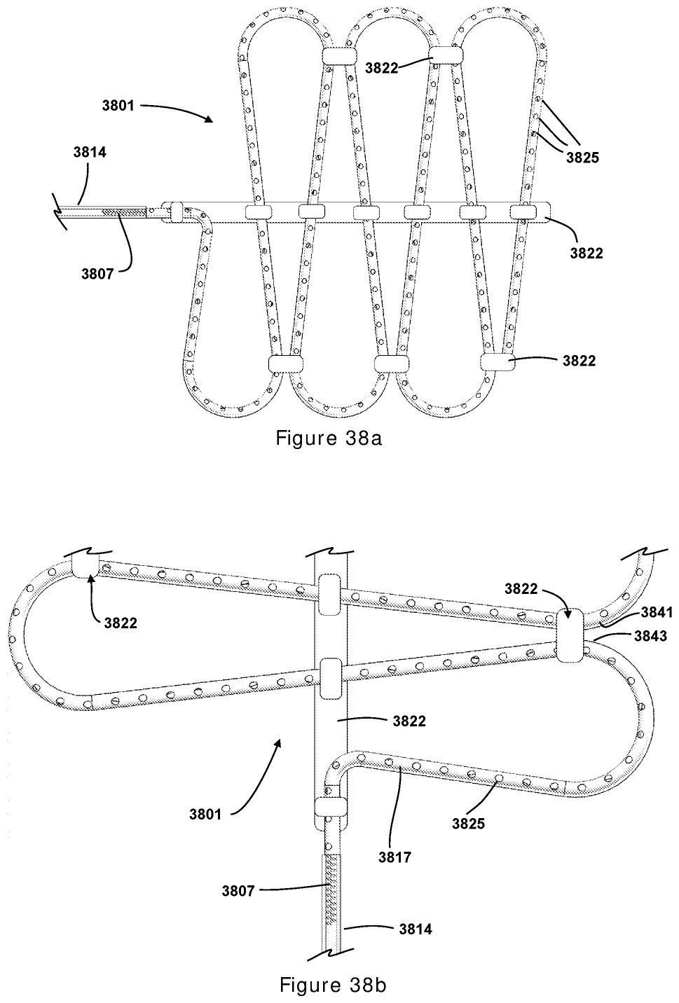

The single channel device may comprise webs or tabs 3822, for example between successive channel bends, to hold the channel in a desired configuration and improve ease of implanting the device as shown in FIGS. 38a and 38b. Preferably the device is configured and arranged so that one or more portions of the channel are positioned near a periphery of the treatment site. In the embodiments shown, the single channel device has a constant channel diameter, but in alternative embodiments, the channel diameter may vary, for example it may be tapered to be narrower at one end.

The type and size of device will be selected based on the characteristics of the treatment site. For example, a branched embodiment may be suitable for a treatment site having a relatively large surface area. In some instances, it will be desirable for the configured device to have a generally wide shape so that the channel or channels for fluid flow spread across the area of the treatment site to the greatest extent possible. In other instances, the shape of the sheets may be long and narrow, for example to lie just underneath a surgical incision line.

Optionally, the device may be temporarily held in its desired configuration by a removable positioning instrument or device that can adjust the shape of the device to suit the area of the treatment site while it is being implanted and secured in place.

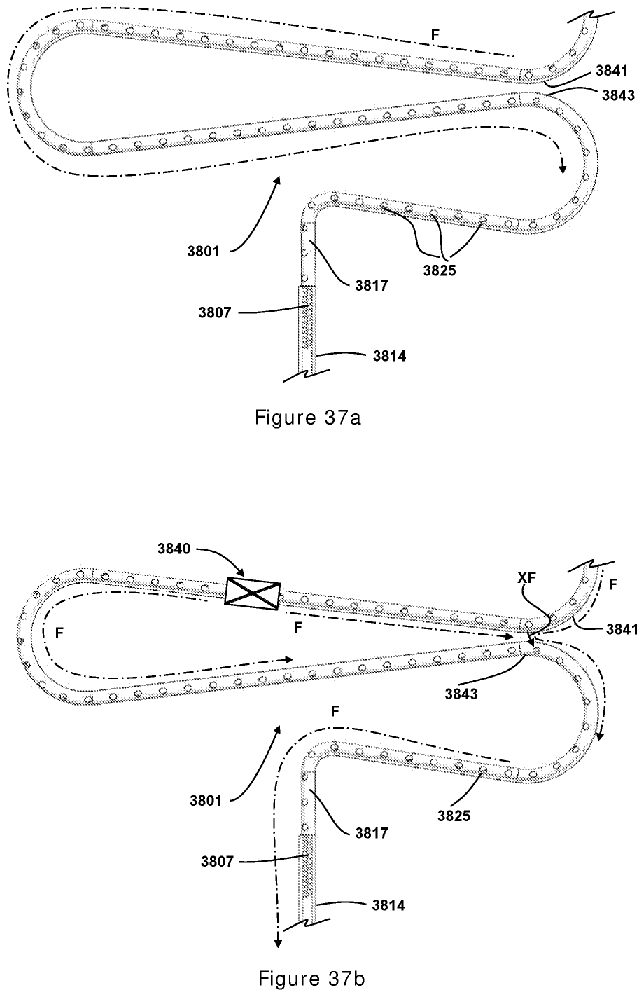

Optionally, one or more channels or the device may be arranged so to provide one or more alternative flow paths in the case that a channel or the device becomes blocked. FIGS. 37a to 38b illustrate an exemplary device with a single channel, configured in a sinuous manner with portions of the channel 3841, 3843 at adjacent bends arranged in close proximity. Referring to FIG. 37b, if the channel experiences a localised blockage 3840 along the primary flow path F, fluid can flow between the portions of the channel 3841, 3843 at adjacent bends to establish an alternative flow path XF.

The device has a port 111 in fluid communication with the channel or channels of the device, so that fluid that drains into any one of the channels will flow towards and out of the port 111. For a device having a branched structure such as those in FIGS. 4 and 12a to 18a with multiple secondary channels 109b, 1309b, 2209b, it will be appreciated that the secondary channels will converge into a primary channel 109a, 1309a, . . . , 2209a, upstream of the port, with the port being located on the primary channel.

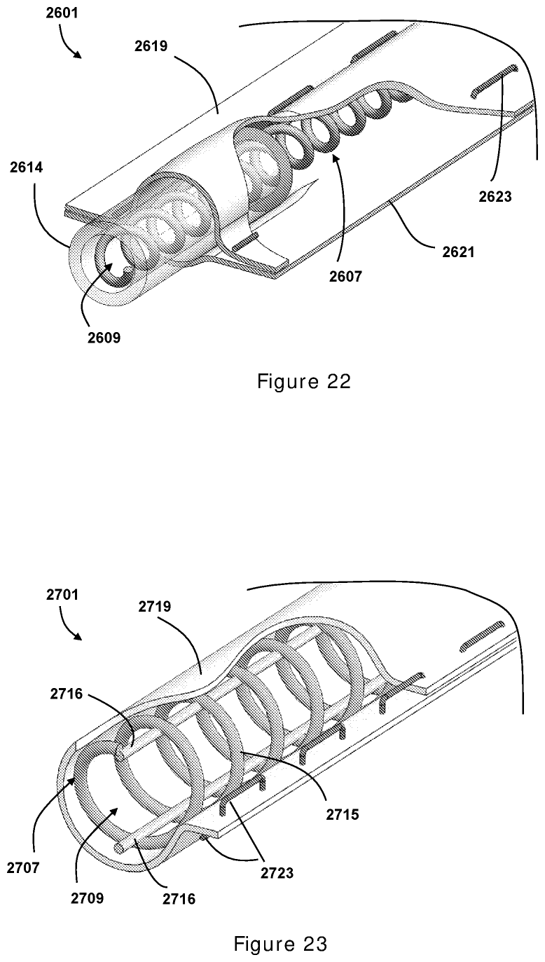

The port may be configured for location internally in a patient or for location externally, for example on the exterior surface of the patient's skin or otherwise the exterior of the patient's body close to a surgical opening in the body. In the case of an internally located port, when in use, the main structure of the device will be located at the treatment site and the port will be located internally within or alternatively near an edge of the treatment site, or conversely positioned at to a remote location elsewhere in the body. The port 111 may merely consist of an opening at the end of the truss or channel, for communication with a conduit 14 from the negative or positive pressure source 13. In some embodiments of the device, 2501, 2601, 3101, the truss 2507, 2670, 3107 extends beyond the flexible sheet or sheets, and for receipt by the conduit 2514, 2614, 3114 (see FIGS. 21, 22, 27a and 27b) to couple the conduit to the channel. When attached to the truss, the conduit may abut the edge of the flexible sheeting, as shown in FIGS. 21, 27a and 39a to 40c, or it may extend under the sheeting (FIG. 22). Alternatively, the port may comprise features to enhance the coupling between the conduit and the device. For example, the shape, diameter, and/or the construction of the device truss may alter adjacent to the port. Referring to FIG. 27b, as one example, the truss 3307 may include a length 3346 adjacent to the port, having an increased diameter to form a releasable connection with the supply conduit 3314, in which the supply conduit 3314 is received internally into the truss 3307. The truss pitch may change in this region 3346 to ensure the connection has the appropriate mechanical properties, for example, the required increase in strength and rigidity. The truss 3307 preferably includes a transition region 3345 in which the change in pitch and change in diameter are gradual.

Alternatively, the apparatus 3901 may comprise a portion 3946 of truss 3907 extending beyond the flexible sheet or sheets 3919 to be received by a conduit 3914 as shown in FIGS. 39a to 40a, to releasably couple the device 3901 to the conduit 3914. In this exemplary embodiment, the conduit 3914 comprises two internal lumens—a primary lumen and a secondary lumen 3947. The internal wall or baffle separating the primary and secondary lumens 3947 may terminate adjacent the end of the coupling portion 3946 of the truss 3907. However, the conduit 3914 preferably includes a flow directing feature forward of this point, for example in the form of a recess/channel in the conduit wall, or a lip or baffle, to direct fluid from the device channel 3909 into the secondary limen 3947 to reduce the occurrence of blockages. The secondary lumen may be useful for the instillation of fluids via the device 3901 to the treatment site, or to facilitate the measurement of parameters such as pressure or temperature at the treatment site.

FIGS. 40a to 40c illustrate the process of removing the conduit 3914 from the truss in this exemplary embodiment. As shown in FIG. 40a, when the conduit 3914 is coupled to the truss 3907, the portion of the conduit overlapping with the truss 3946 expands to fit over the truss such that the diameter of the conduit is greater in the region where it overlaps with the truss. A plurality of slits 3948 are provided in the wall of the conduit in the coupling region of the conduit to facilitate expansion of the conduit 3914 over the truss. The slits extend longitudinally along the conduit wall, and may be provided around the perimeter of the conduit or only in particular regions of the conduit, for example at top and bottom regions, with more slits provided in regions where more expansion of the conduit wall is desirable. These slits 3948 are useful to facilitate coupling between the oval truss 3907 and a conduit with a different cross-sectional shape, for example a cylindrical conduit. The expanded portion of the conduit applies a compressive force to the truss 3907 to form a secure connection.

As illustrated in FIGS. 40b and 40c, to remove the conduit 3914 from the truss 3907, the conduit is pulled in the longitudinal direction, off the truss. The slits 3948 assist with removal as the conduit walls slide off the end of the truss, the slits 3948 in the wall close and the conduit walls contract until they revert to substantially their original shape and dimensions. The conduit walls preferably comprise a resilient material such as silicone, to assist with reverting of the expanded conduit portion to its original dimensions and to ensure a secure connection.

It will be appreciated that other methods of coupling the device to the supply conduit are appreciated and envisaged, including additional retention features. For example, an interior surface of the conduit could may be threaded or have protrusions/detents for additional engagement with the truss to prevent unintended disconnection. A secondary retention method may include utilising a loop of thread or suture passed down a lumen of the conduit and threaded through the interface of the truss members and the conduit to provide a secure connection which can be simply released by pulling on loop of thread to release the connection.

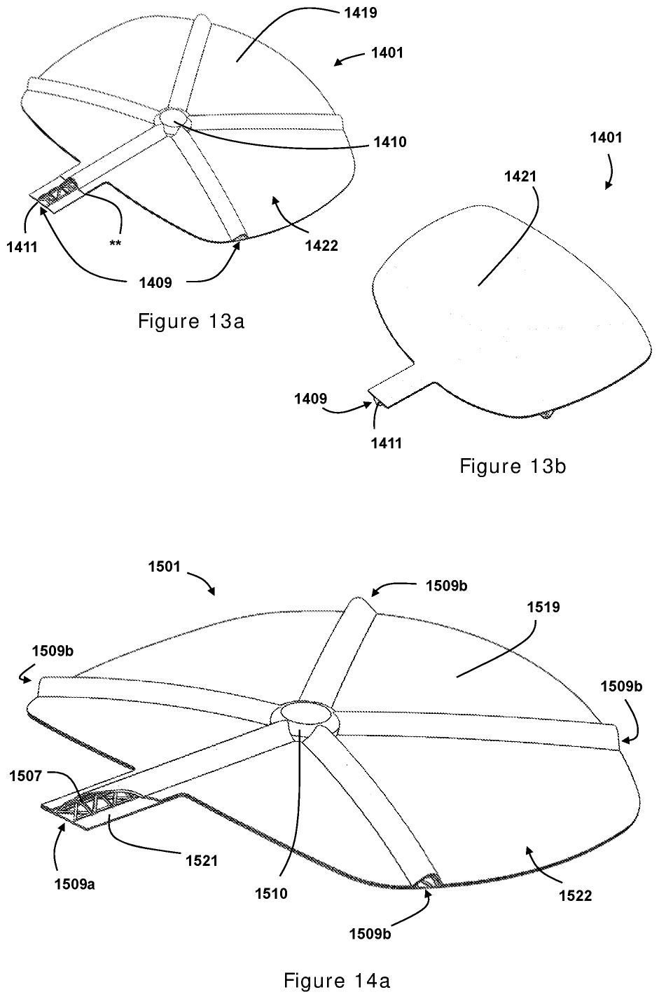

The flexible sheet or sheets may be cut away adjacent to the port as shown in FIGS. 13 and 18, in embodiments where the port is intended to be located externally and where that respective portion of truss will not be surrounded by tissue.

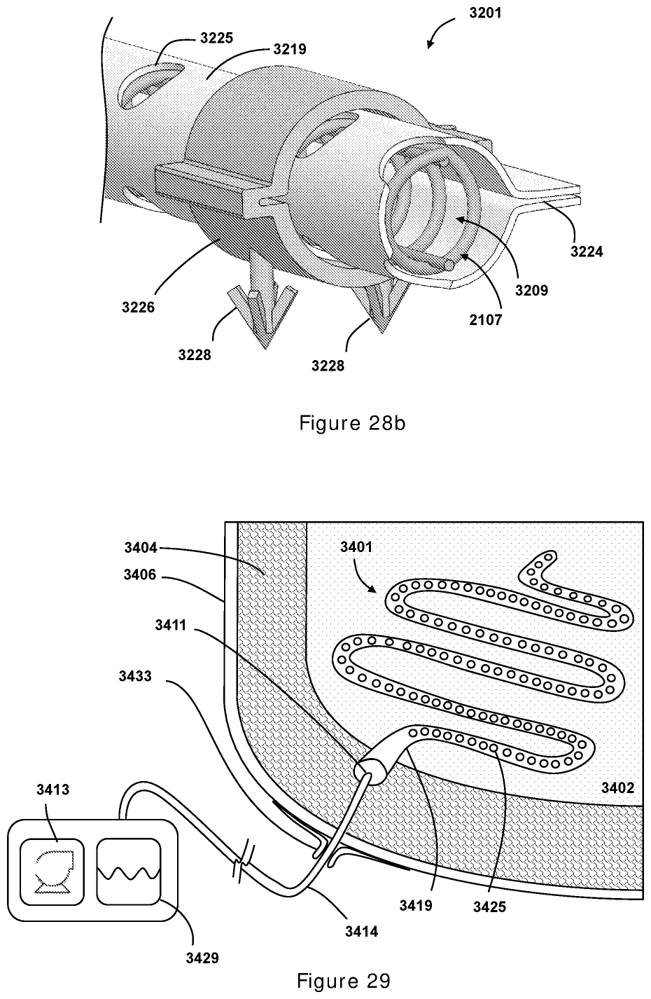

The device may comprise one or more features to secure the device relative to soft tissue. FIGS. 28a and 28b illustrate an embodiment having an absorbable locking component 3226, for example comprising a cuff of polymeric material wrapped around the device 3201. Tissue retention barbs 3228 protrude from the cuff, for securing the device 3201 to soft tissue at the treatment site.

An externally positioned port may have a similar form to those described above in relation to an internally positioned port. Advantageously, when the function of fluid drainage of fluid delivery is complete, the conduit can be decoupled from the externally positioned port, and the port can be inserted into the body through the surgical opening and the opening surgically closed. As the entire device is formed from bioresorbable materials, the port will then be absorbed or remodelled by the body along with the device over time. Alternatively, the port of the device may be cut off or otherwise removed from the device and the surgical opening then surgically closed.

The device described above is intended for use in a system for draining fluid from a treatment site or delivering fluid to a treatment site in the body of a patient. Exemplary systems are shown in FIGS. 2a to 4, 29 to 32, 35a to 36b and 41 to 42. The system comprises a conduit 3414 that is releasably coupled to either the port 3411 of the device 3401 or to a fluid impermeable dressing, and to a reservoir 3429 located external to the body of the patient, the reservoir in fluid communication with the conduit 3414 for receiving fluid from the conduit. Alternatively or additionally the system may have a reservoir 3437 holding a treatment fluid for delivering fluid to the conduit 3414. A source of pressure 3413 is coupled to the conduit 3414 for delivering positive pressure or negative pressure to the device 3401.

In some embodiments, the port 3411 may be coupled to an impermeable dressing 3433 located on the exterior surface of the skin 3406 which provides an airtight hermetic seal around the incision of the skin and an alternative means to which a conduit is releasably coupled to the dressing. One exemplary system is schematically illustrated in FIG. 2a which provides a cross-sectional view of an abdominal cavity 108 where a device 101 has been placed adjacent to a muscle 103 to remove fluid from a seroma 105. The port 111 of the device 101 is shown to be proud of the exterior surface of the skin 106 and is covered and connected to an impermeable hermetic dressing 112 which is releasably coupled to a conduit in the form of a tube 114 in connection with a negative pressure or positive pressure source 113. The device 101 and truss 107 continue from the exterior surface of the skin 106, through the subcutaneous tissue 104, to the treatment site 102 where the device 101 is in contact with both the seroma 105 or dead space and the muscle tissue 103. The channel 109 within the device 101 provides fluid communication between the seroma 105 and the port 111 of the device 101. In alternative embodiments, the port 111 may instead be internal, for example provided near an edge of the treatment site 102.

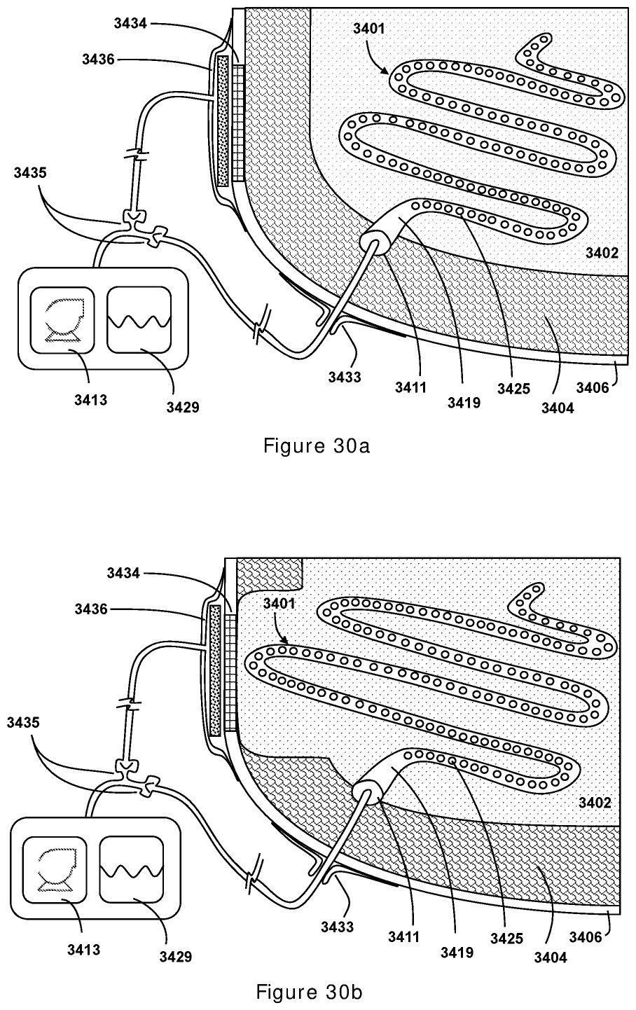

With reference to FIG. 29, alternatively, the coupling between the conduit 3414 and the port 3411 may be provided internally in the patient, as shown in the embodiments of FIGS. 29 to 32. In that embodiment, the device 3401 is positioned beneath a layer of subcutaneous or subcutaneous and muscle tissue 3404. The pressure source 3413 for the system may also be utilised to apply pressure to a wound dressing 3436, for example a dressing over a surgical incision 3434. One such system is illustrated in FIGS. 30a and 30b, where connectors 3435 couple respective conduits for the dressing 3436 and the drainage device 3401 to the pressure source 3413 and conduit 3429.

The source of pressure 3413 may be capable of delivering negative pressure to the device 3401 so that fluid is drained from the treatment site 3402 into the device 3401 and transferred through the conduit 3414 to the reservoir 3429, or may be capable of delivering positive pressure to the device so that fluid in the reservoir is transferred through the conduit into the device and to the treatment site. The fluid flow path is indicated in for the embodiments shown in the drawings by flow arrows F.

The source of pressure will typically be a pump for pumping fluid from the reservoir into the device 3401 for delivery to the treatment site or a vacuum pump 3413 for applying negative pressure to drain fluid from the treatment site 3402. The pump may be manually operated, for example using a squeeze bulb, or may be electronically controlled for more precise delivery of fluid to the site.

In a system where fluid is being delivered to the treatment site, the fluid to be delivered may contain one or more nutrients, Towable fluids' such as Thixotropic gels or highly viscous fluids that can still be transported via a conduit, cell-suspensions therapeutic agents for promoting wound healing. The device described herein may advantageously be customised to adjust the duration for which the device is functional in-situ for any given application. For example, by adjusting wall thicknesses, or the thickness or density of truss members.

### III. Method of Manufacture

FIGS. 33a to 33d illustrate steps of an exemplary method of forming a truss having two helical truss members 3515a, 3515b. In a first step shown in FIG. 33a, a first truss member 3515a in the form of suture or other bioresorbable polymeric filament is clamped at one end by a clamp 3544 and wound around a rod-like mandrel 3530 in a helical manner at a first pitch length P1. Two elongate bracing members 3516 are then also clamped at their ends by the clamp 3544, and laid over the helical truss member, along opposing sides of the mandrel. A second truss member 3515b is then clamped by the clamp 3544, wound around the first helical member 3515a, bracing members 3516, and the mandrel 3530, as shown in FIG. 33c. The mandrel 3530 or the environment is then heated causing the bracing members to fuse to the first helical member at the points where they overlap. The truss 3507 is allowed to cool, setting the shape of the truss members, then the clamp 3544 and mandrel 3530 are removed leaving the hollow truss 3507 as shown in FIG. 33d. It will be apparent that the order of the method steps may vary, and that not all steps are necessary.

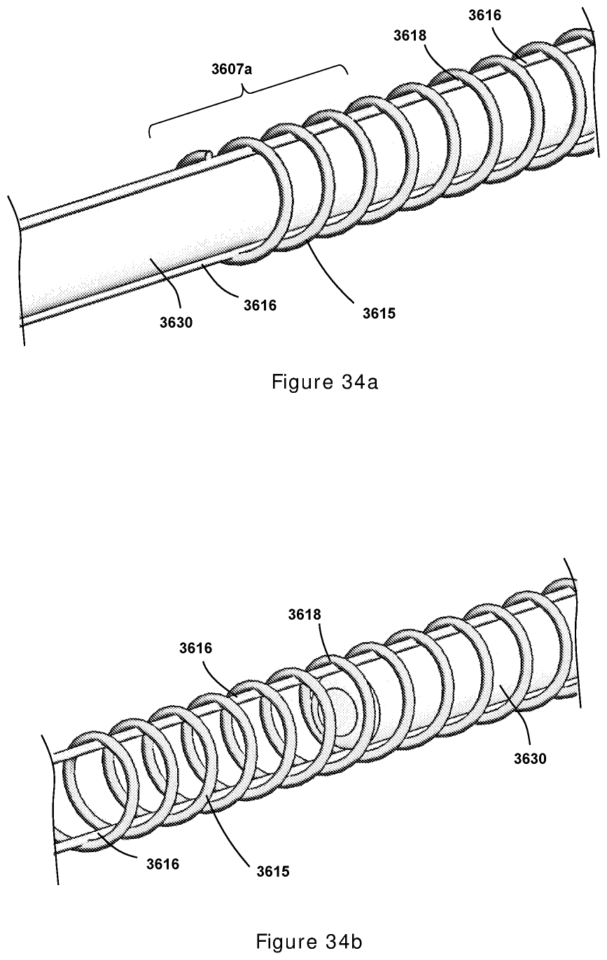

FIGS. 34a and 34b illustrate an alternative exemplary method in which the truss is continuously manufactured. Two elongate bracing members 3616 are fed along opposing sides of the mandrel 3630, and a truss member 3615 is then wound around the bracing members 3616, as shown in FIG. 34a, with a portion 3607a of the members attached to the bracing members 3616 by the local application or heat, or clamped or otherwise secured relative to the mandrel 3630. As the truss member 3615 is wound, heat is locally applied near the vertices 3618 of the truss members 3615, 3616 to bond the truss member and bracing members together. The truss 3607 is allowed to cool, setting the helical shape of the truss member 3615 before the truss is indexed off the mandrel 3630 and the process continues.

In alternative embodiments, such as those devices having the truss and sheet arrangements of FIGS. 5a to 11 b, a length of bioresorbable resilient thread or tape such as suture may be woven or sewn into or through at least one flexible sheet to form the truss. For example, by machine sewing a zig-zag type stitch over a rod to provide a three-dimensional form creating a channel. The upper and lower threads used to create the machine sewn zig-zag stitch may be of different gauges or thicknesses to facilitate interlocking of the stiches. Embodiments constructed using a zig-zag stitch may include an additional lower sheet 221, 321 (see FIGS. 5a and 5b) to prevent tearing during manufacture.

### IV. Materials

The device of the invention is formed from bioresorbable materials. Typically, two types of bioresorbable material will be used, one for the flexible sheets and any webs and one for the truss.

In some embodiments of the invention, the flexible sheet(s) are formed from ECM. The ECM sheets are typically collagen-based biodegradable sheets comprising highly conserved collagens, glycoproteins, proteoglycans and glycosaminoglycans in their natural configuration and natural concentration. ECM can be obtained from various sources, for example, dermis pericardial or intestinal tissue harvested from animals raised for meat production, including pigs, cattle and sheep or other warm blooded vertebrates.

The ECM tissue suitable for use in the invention comprises naturally associated ECM proteins, glycoproteins and other factors that are found naturally within the ECM depending upon the source of the ECM. One source of ECM tissue is the forestomach tissue of a warm-blooded vertebrate. The ECM suitable for use in the invention may be in the form of sheets of mesh or sponge.

Forestomach tissue is a preferred source of ECM tissue for use in this invention. Suitable forestomach ECM typically comprises the propria-submucosa of the forestomach of a ruminant. In particular embodiments of the invention, the propria-submucosa is from the rumen, the reticulum or the omasum of the forestomach. These tissue scaffolds typically have a contoured luminal surface. In one embodiment, the ECM tissue contains decellularised tissue, including portions of the epithelium, basement membrane or tunica muscularis, and combinations thereof. The tissue may also comprise one or more fibrillar proteins, including but not limited to collagen I, collagen III or elastin, and combinations thereof. These sheets are known to vary in thickness and in definition depending upon the source of vertebrate species.

The method of preparing ECM tissues for use in accordance with this invention is described in U.S. Pat. No. 8,415,159.

In some embodiments of the invention, sheets of polymeric material may be used. The polymeric material may be in the form of sheet or mesh. Synthetic materials such as polyglycolic acid, polylactic acid and poliglecaprone-25 will provide additional strength in the short-term, but will resorb in the long term. Alternatively, the polymeric material may be a natural material, or derived from a natural material, such as a proteins (e.g. collagen), a polysaccharides (e.g. alginate), and a glycoprotein (e.g. fibronectins).

It will be understood that the truss members forming the truss will be formed from a material that has a degree of flexibility to allow the device to conform to the contours of the treatment site, and will have sufficient structural strength and integrity to hold the two surfaces apart and thereby allow channels to form. The structural integrity of this material and resulting shape will also provide a means for the fluid flow channel to be reinstated should the device be kinked or crushed in any circumstance. For example, the truss members may comprise a length of suture, thread, cord, or tape made from a bioresorbable material such as polyglycolic acid (PGA), polylactic acid (PLA), polyglycolic-polylactic copolymers, P4HB (Poly-4-hydroxybutyrate), polycaprolactone or polydioxanone.

### V. Delivery of Bioactive Materials

Any desirable bioactive molecules can be incorporated into the ECM or polymeric material or the truss member material itself. Suitable molecules include for example, small molecules, peptides or proteins, or mixtures thereof. The bioactive materials may be endogenous to ECM or maybe materials that are incorporated into the ECM and/or polymeric material during or after the grafts manufacturing process. In some embodiments, two or more (e.g. 2, 3, 4, 5, 6, 7, 8, 9, 10 or more) distinct bioactive molecules can be non-covalently incorporated into ECM or polymer. Bioactive molecules can be non-covalently incorporated into material either as suspensions, encapsulated particles, micro particles, and/or colloids, or as a mixture thereof. Bioactive molecules can be distributed between the layers of ECM/polymeric material. Bioactive materials can include, but are not limited to, proteins, growth factors, antimicrobials, and anti-inflammatories including doxycycline, tetracyclines, silver, FGF-2, TGF-B, TGF-B2, BMR7, BMP-12, PDGF, IGF, collagen, elastin, fibronectin, and hyaluronan.

### VI. Surgical Placement

The surgical placement of the device is best shown in FIG. 4 where the device 101 is shown to be secured to the muscle 103, for example, within the abdomen. The port 111 of the device 101 is shown to exit out of an incision 142 which is separate from the primary surgical incision 134 and allows the device 101 to be placed on to the exterior surface of the skin 106. The structure of the device 101 used to separate the device layers 119, 121 is visible on the exterior surface of the skin 106 and in this particular view is shown to pass through the subcutaneous layers of tissue 104 where the lower surface of the device 101 is secured to the muscle 103 beneath.

FIG. 2a illustrates an example of where the port 111 of the device 101 is covered with an impermeable hermetic skin dressing 112 which provides an air tight seal around the device and the exit incision 142 shown in FIG. 4. The impermeable hermetic dressing also provides a means to releasably connect a tube 114 to a source of negative or positive pressure 113 in order to exchange fluid from the source or the treatment site 102. The primary surgical incision is typically covered with either a breathable or impermeable dressing (not shown) depending on the clinical application.

Alternatively the device could be placed at the bottom of an open wound and used in conjunction with a dressing. FIGS. 41 and 42 are examples of placement in an open wound utilising a device 3901 having an oval truss 3907 and channel 3909. The device 3901 is positioned at or near the surface of exposed muscle 4003, within the subcutaneous tissue 4004 to create a drainage channel 3909. Negative pressure may be applied to the device 3901, to suction fluid from the channel 3909 as the wound heals. in the example 4034 of FIG. 41, an air occlusive dressing 4039 is applied over the remaining exposed area of the wound to permit the application of negative pressure to the treatment site via the device 3901 while also protecting the exposed surfaces of the wound. Optionally, a negative pressure wound dressing 4139 may be applied over the wound, in fluid communication with the device 3901 as shown in FIG. 42. In the embodiment shown in FIG. 42, a foam dressing is placed in the wound, over the device 3901, with a sealing dressing 4112 over the skin and surrounding periwound area.

### VII. Instillation of Treatment Fluids

The ability to controllably instil and dose flowable and cell-based fluids to treatment sites following surgery is desirable in many clinical procedures following the surgical excision of cancerous tissue or where ongoing infection is a concern. The ability to precisely control various parameters such as the dose concentration, contract time, dose volume and site at the treatment site also offers an advantage over existing drug eluting or dosed implant devices which often rely on the degradation properties of the material for a dosage profile.

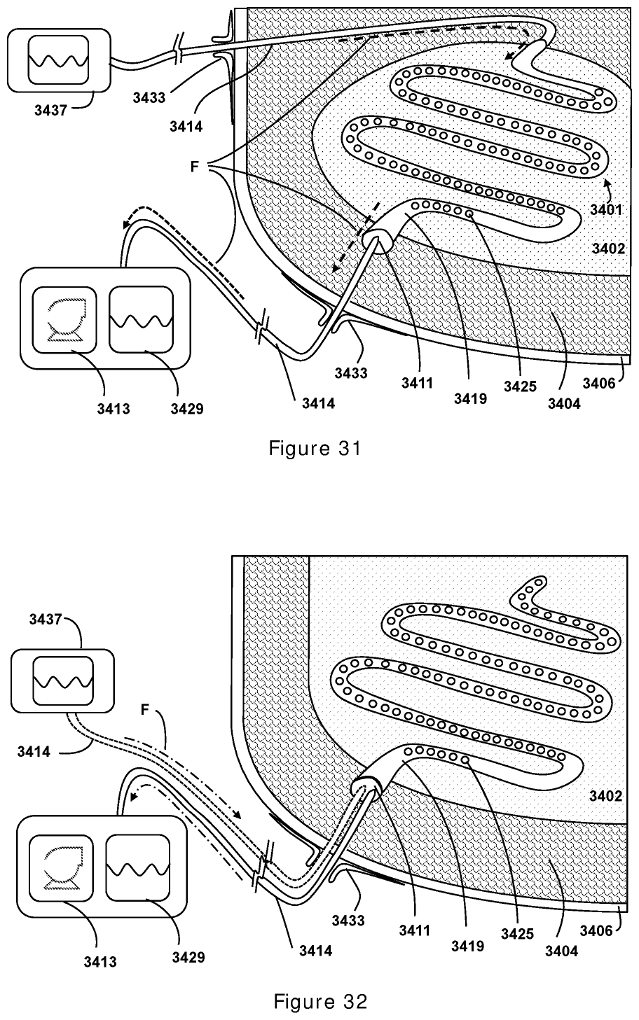

FIGS. 31, 32 and example 5 below describe embodiments where treatment fluid can be instilled to treatment site in precise and targeted way. The fluid may comprise flowable gels derived from ECM, hyaluronic acid, growth factors to aid healing, to antimicrobial drugs for the treatment of infection, analgesic drugs such as fentanyl or morphine for pain relief and anti-inflammatory drugs such as ketorolac or diclofenac, for example, although other fluids are envisaged and will be apparent to a skilled person.

Instillation of autologous or allogenic cell-based therapies containing either platelet rich plasma, stem cells, stromal cells, keratinocytes, lymphocytes, bone marrow aspirate, serum and dendritic cells could aid in the repair and healing of wounds.

For example, the instillation of intestinal stem cells could help in the treatment of inflammatory bowel disease, while the instillation of pancreatic islet cells following partial or complete a pancreatectomy could aid in the repair and regeneration of damage tissues.

The instillation of chemotherapeutic drugs could also aid in the localised treatment of cancerous cells that may not be operable, or could be used as an overall treatment plan following excision of cancerous tissue.

### EXAMPLES

### Example 1: Closing Subcutaneous Tissue Dead Space Below a Surgical Incision

The management of a subcutaneous tissue under a closed surgical incision can be clinically challenging in procedures which involve a large amount of adipose (fat) tissue. Adipose tissue is known to possess poor mechanical strength in its ability to retain suture and the elevated distance between the skin and the underlying muscle can lead to the formation of a dead space that allows the collection of fluids post-surgery which can lead to later complications such as wound dehiscence and surgical site infections. The example given below demonstrates how this device may be utilised to eliminate the surgical dead space beneath a surgical incision.

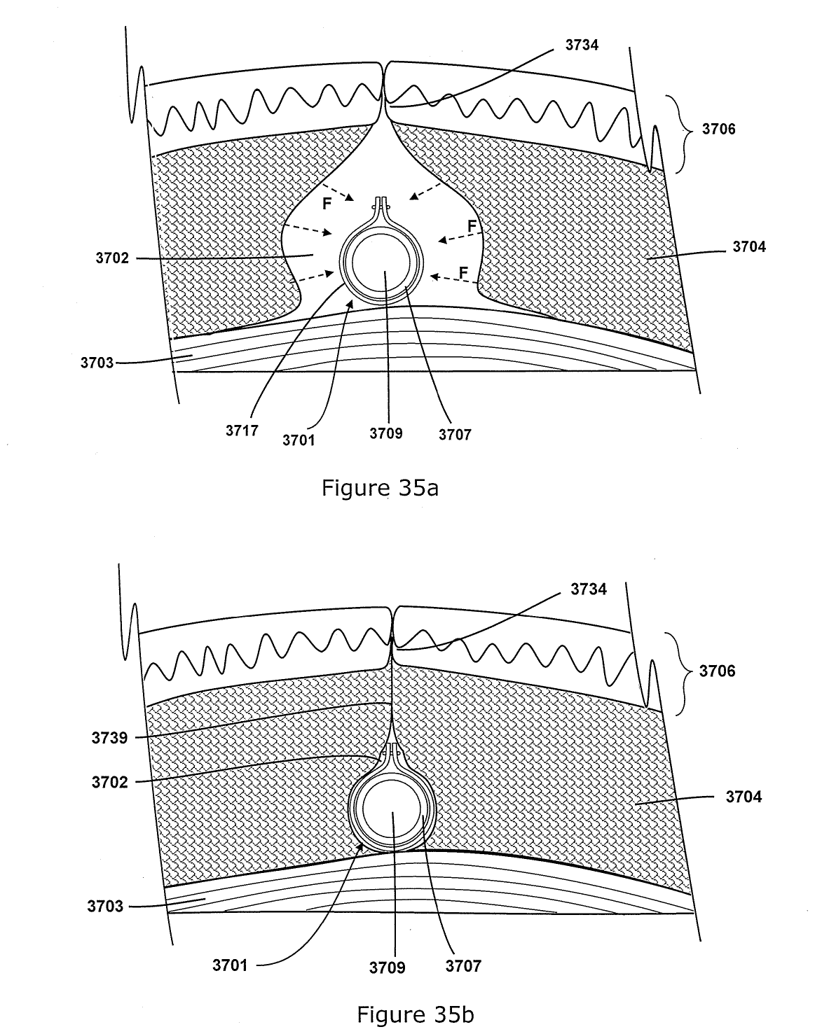

The surgical placement of the device is best shown in cross-sectional views of FIGS. 35a and 35b where the device 3701 is shown to be secured to the muscle 3703 at the treatment site 3701 below a skin incision 3734. The implanted treatment device 3701 is shown to comprise a hollow truss 3707 surrounded by a single layer of ECM or polymeric material to define a channel 3709 to allow the passage of fluids between the treatment site 3702 and a source of negative pressure coupled to the device (coupling not shown). Once negative pressure or suction is applied, the fluid F from the treatment site 3702 is drawn towards the device 3701, resulting in reduction and closure of the dead space within the subcutaneous tissue to create apposition 3739 of the two opposing faces of previously separated tissue 3704, as illustrated in FIG. 35b.

### Example 2: Dual Management of a Surgical Incision

Topically applied wound treatment devices which apply negative pressure to the surface of a primary surgical incision have become widely adopted for the prevention of surgical complications such as wound dehiscence and surgical site infections. These topically applied devices primarily aid healing by providing a secondary mechanical retention to reduce the tensile force on the primary suture line and by covering and removing excess exudate from the skin to prevent maceration and infection.

While these devices have demonstrated effectiveness at supporting the healing of the skin incisions, they are unable to effectively manage the dead space of deeper subcutaneous tissues particularly those that have been subjected to a large amount of undermining, separation or excision which often require a greater amount of time to heal than compared to a skin incision. In these scenarios, a combined system of the implanted treatment device 3701 and a topically applied wound treatment device 3736 may be utilised to eliminate dead space at an internal treatment site while managing the healing of a surgical skin incision 3734.

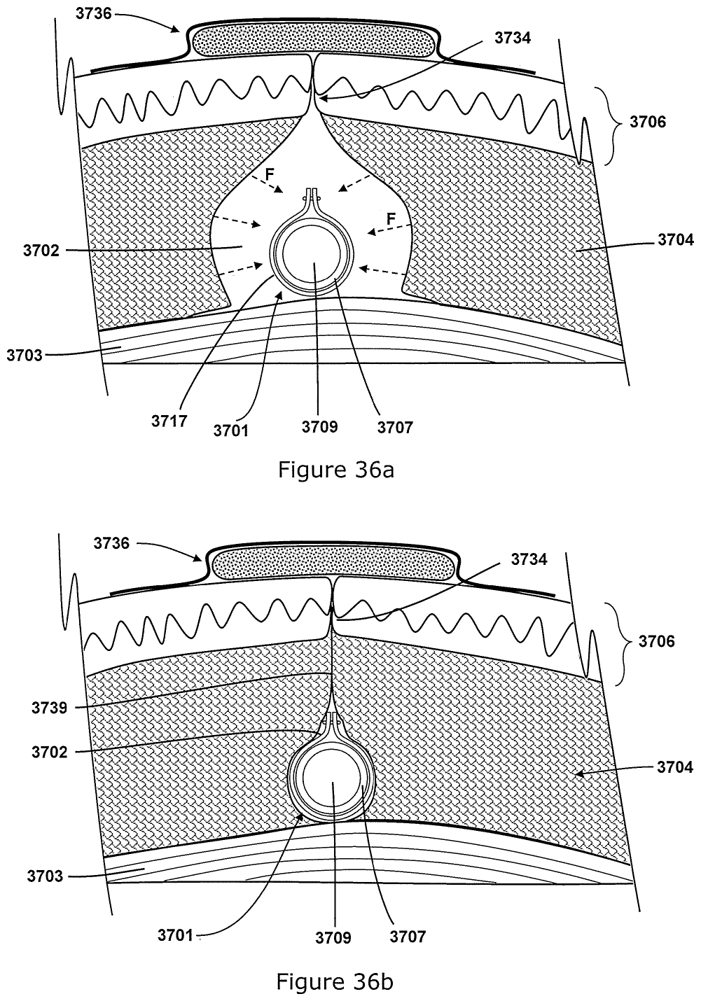

The surgical placement of a combined topical and implanted treatment system is best shown in the cross-sectional views of FIGS. 36a and 36b. The device 3701 is shown to be secured to the muscle 3703 at the treatment site 3702 below a skin incision 3734. The implanted treatment device 3701 is comprises a truss 3707 surrounded by a single layer of ECM or polymeric material to define a channel 3709 to allow the passage of fluids between the treatment site 3702 and the source of negative pressure. A topically applied wound treatment device 3736 is positioned over the skin incision 3734 to allow for simultaneous treatment to the incision wound.

Once negative pressure or suction is applied to the implanted treatment device 3701 the fluid F from the treatment site is drawn towards the device 3701 reducing and closing the dead space within the subcutaneous tissue 3704 to create apposition 3739 of the two opposing faces of previously separated tissue 3704, as shown in FIG. 36b.

A schematic of a combined treatment system is additionally shown in FIGS. 30a and 30b, in which an implanted treatment device 3401 has been positioned and secured at a treatment site 3402 within the body with a topically applied wound treatment device 3436 applied to the skin incision 3434. FIG. 30a shows the treatment site 3402 to be beneath a layer of subcutaneous tissue 3404 which could be at a site comprising either adipose tissue, muscle, bone, tendon or any combination of these tissues.

With reference to FIG. 30b the treatment site 3402 could comprise a dead space within subcutaneous tissue 3404 positioned either beneath a skin incision or a primary skin incision 3434 which has been closed with either sutures or staples. In both FIG. 30a and FIG. 30b the implanted treatment device 3401 and the topically applied wound treatment device 3436 have been coupled to tubes which are connected to each other via a tube connector 3435 to convey fluids to and from the wound via the negative or positive pressure supplied by the pressure source 3413. The pressure source 3413 also contains a suitable reservoir 3429 to store fluids extracted from the treatment sites or optionally fluids for installation to a treatment site.

In both figures the implanted treatment device 3401 is a single channel device arranged in a sinuous configuration to allow the device 3401 to effectively deliver treatment over a large area. However, other device types or configurations may be utilised.

### Example 3: Method of Manufacturing a Truss Component

One example of manufacturing the device truss component is described generally above in relation to FIGS. 33a to 33d, with truss members 3515a, 3516 and 3515b clamped at their first end, tightly wound around the central mandrel 3530, and clamped at their opposing ends.

At this point, the entire assembly is placed into the oven at a temperature of about 120° C. for approximately 5 minutes to allow all the intersecting vertices 3518 of the truss members 3516, 3515a, 3515b to bond together. Once adequate bonds have has formed, the assembly is removed from the oven and allowed to cool before both clamps 3544 are removed and the central forming mandrel 3530 is removed to leave the device truss 3507 as a single resilient yet flexible and pliable component as shown in FIG. 33d.

In this example the second (outer) truss member 3515b is wound with a continuous pitch that differs to the pitch of the first truss member at a ratio of 3.5:1.

The truss members in this example comprise USP size #0 bioresorbable polydioxanone monofilament suture material which is approximately 0.4 mm in diameter, but this method could be used for any diameter of suture with any material type. The oven temperature and the time of heat application will vary for different embodiments, for example, depending on the size and material properties of the truss members, and the number of truss members. For this example, the choice of monofilament suture is made to provide the suitable rigidity required to form the resilient yet pliable final truss structure, but either monofilament or braided or any combination of the two types of filament could be used depending on the structure and truss properties required.

### Example 4: Method of Manufacturing a Truss Component in a Continuous Manufacturing Method

An alternative method of manufacturing the truss in a continuous process is given below. With reference to FIG. 34a, two longitudinal ‘bracing’ truss members 3616 are fed along a central forming mandrel 3630. A first truss member 3615 is attached along an end portion 3607a to the two longitudinal ‘bracing’ truss members 3607a by the application of localised heat. Subsequent cooling of this area completes the fusing of this localised section to anchor the truss members together to allow for a rotating winding feeder to continuously wrap the first truss member 3615 around the central mandrel 3630 and bracing truss members 3616 while the member is being fed along the central forming mandrel 3630.

As the first truss member 3615 is wound, heat is locally applied to a zone of the assembly to bond intersecting vertices 3618 of the truss members 3615, 3616. Once adequate heat has been applied, the formed truss 3607 is cooled and indexed off the central mandrel 53 as shown in FIG. 34b to allow the continuous process to proceed.

### Example 5: Treating a Wound Site by Supplying Fluid to and Removing Fluid from a Treatment Site

The ability to administer drugs and fluids to a targeted treatment site within the body has become an important tool within the field of medicine particularly for the treatment of pain, localised infections or diseases. While routine administration of drugs is common for many patients globally the length of treatment can widely vary from a short duration to patients with life dependency.

One aspect of the device disclosed herein is the ability to couple the implanted device to a source of treatment fluids such as antibiotic drugs, flowable gels, cell-based fluids and pain relief drugs for a prescribed contact time. A schematic representation for such a treatment system is best shown in FIG. 31. In this example, the treatment site 3402 is positioned in an isolated localised area beneath either subcutaneous tissue 3404, or a combination of both subcutaneous tissue 3404 and muscle tissue, with the implant treatment device 3401 releasably connected to a source of treatment fluid 3437 via a conduit 3414 at one end of the treatment device with the opposite end of the treatment device 3411 releasably connected to source of negative pressure 3413.

The implanted treatment device 3401 is also shown to contain several apertures 3425 in the channel walls to allow the passage of fluid out of the device, through the device surface 3419. The positioning of the channel apertures 3425 is particularly important for the controlled administration of drugs to the desired treatment site 3402. While the implanted treatment device 3401 is shown to have channel apertures 3425 along much of the length of the device 3401, the position and frequency of these apertures can be adjusted to suit the site of treatment 3402.

In this example the source of negative pressure 3413 could be operating in either a continuous, intermittent, constantly varying or discontinuous mode where the applied negative pressure could range from 0 mmHg to 200 mmHg or cycle between any prescribe levels during operation. The instillation of drugs can be administered by opening a valve on the treatment fluid reservoir 3437, or injecting fluids into the treatment reservoir 3437, where the source of negative pressure would draw the treatment fluid towards the source of negative pressure 3413. The time of which the drug is in contact with the treatment site can be controlled by the operation of negative pressure at the pressure source 3413, which could be stopped to hold the drug statically within the channels of the device 3401.

Alternatively the administration of drugs could be controlled by injecting or connecting the treatment fluid reservoir 3437 to a source of sterile saline or other fluid to purge the line clear of any treatment drugs.

Any reference to prior art documents in this specification is not to be considered an admission that such prior art is widely known or forms part of the common general knowledge in the field.

As used in this specification, the words “comprises”, “comprising”, and similar words, are not to be interpreted in an exclusive or exhaustive sense. In other words, they are intended to mean “including, but not limited to”.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A bioresorbable device for implantation at a treatment site in the body of a patient for draining fluid from the treatment site or delivering fluid to the treatment site, the device comprising:
a bioresorbable resilient truss for holding two tissue surfaces spaced apart, thereby defining a channel into which fluid from the treatment site can drain or from which fluid can be delivered to the treatment site; and
a port in fluid communication with the one or more channels and being connectable to a source of negative pressure or positive pressure.

2. A device as claimed in claim 1, wherein the truss comprises a flexible elongate truss member that is curved and located along a wall of the channel

3. (canceled)

4. A device as claimed in claim 2, wherein the truss member is substantially helical.

5. A device as claimed in claim 2, wherein the truss forms a flexible tube defining the channel.

6. A device as claimed in claim 1, further comprising a plurality of flexible elongate truss members

7. A device as claimed in claim 6, wherein a first one of the truss members is substantially helical with a first pitch length, and a second one of the truss members is substantially helical in a direction opposite to the helical direction of the first truss member and with a second pitch length that is substantially equal to the first pitch length, the first and second truss members being joined together at discrete points.

8. (canceled)

9. A device as claimed in claim 7, further comprising two flexible elongate side truss members, each extending longitudinally along a side of the channel and joined at discrete points to the first and/or second truss members.

10. A device as claimed in claim 1, further comprising a flexible bioresorbable sheet, the sheet forming at least a portion of a wall of the channel, such that the channel is formed between a surface of the flexible sheet and the surface of tissue or bone of the treatment site.

11. (canceled)

12. A device as claimed in claim 10, wherein the flexible sheet is wrapped around the truss.

13. A device as claimed in claim 10, comprising a plurality of apertures in the flexible sheet along a wall of the channel to permit fluid flow into the channel.

14. A device as claimed in claim 1, further comprising two flexible bioresorbable sheets, wherein the channel is formed between facing surfaces of the two flexible sheets.

15. A device as claimed in claim 14, further comprising a plurality of apertures in one or both flexible sheets along a wall of the channel to permit fluid flow into the channel.

16. A device as claimed in claim 10, wherein at least one truss member comprises a length of thread or tape woven or sewn into or through at least one flexible sheet.

17. (canceled)

18. A device as claimed in claim 10, wherein the or each flexible sheet comprises one or more layers of extracellular matrix (ECM) or polymeric material.

19. A device as claimed in claim 18, wherein the ECM is formed from decellularised propria-submucosa of a ruminant forestomach.

20. A device as claimed in claim 19, wherein the ECM contains a bioactive agent selected from the group comprising doxycycline, tetracyclines, silver, FGF-2, TGF-B, TGF-B2, BMR7, BMP-12, PDGF, IGF, collagen, elastin, fibronectin, and hyaluronan.

21. A device as claimed in claim 1, wherein the truss forms an elongate flexible tube defining the channel, and the device comprising one or more joiners holding at least a length of the flexible tube in a sinuous shape.

22.-25. (canceled)

26. A system for draining fluid from a treatment site or delivering fluid to a treatment site in the body of a patient comprising:
(i) a device as claimed in claim 1;
(ii) a conduit releasably coupled to either the port of the device or to a fluid impermeable dressing;
(iii) a reservoir located external to the body of the patient, the reservoir in fluid communication with the conduit for receiving fluid from the conduit or delivering fluid to the conduit; and
(iv) a source of pressure coupled to the conduit for delivering positive pressure or negative pressure to the device.

27. A system as claimed in claim 26, wherein the source of pressure is capable of delivering negative pressure to the device so that fluid is drained from the treatment site into the device and transferred through the conduit to the reservoir.

28. (canceled)

29. A method of draining fluid from a treatment site or delivering fluid to a treatment site in the body of a patient comprising:
(i) implanting a device of claim 1 at the treatment site;
(ii) coupling a conduit to the port of the device, the conduit being connected to a reservoir located external to the body of the patient for receiving fluid from the conduit or delivering fluid to the conduit; and
(iii) delivering negative pressure to the device so that fluid is drained from the treatment site into the device and transferred through the conduit to the reservoir, or so that fluid in the reservoir is transferred through the conduit into the device and to the treatment site.

30. A system as claimed in claim 26, wherein the source of pressure is capable of delivering negative pressure to the device to convey fluids to and from the treatment site.
